(19) United States
(12) Patent Application Publication
(10) Pub. No.: US 2003/0139662 A1
(43) Pub. Date: Jul. 24, 2003

(54) Method and apparatus for detecting, identifying and performing operations on microstructures including, anthrax spores, brain cells, cancer cells, living tissue cells, and macro-objects including stereotactic neurosurgery instruments, weapons and explosives

(76) Inventor: Abraham Neil Seidman, Los Angeles, CA (US)

Correspondence Address: Abraham N. Seidman, P. O. Box 16603, Beverly Hills, CA, 90209, US

(21) Appl. No.: 10/272,685

(22) Filed: Oct. 16, 2002

Related U.S. Application Data
(60) Provisional application No. 60/329,785, filed on Oct. 16, 2001.
(60) Provisional application No. 60/350,454, filed on Oct. 24, 2001.
(60) Provisional application No. 60/336,076, filed on Nov. 23, 2001.
(60) Provisional application No. 60/336,389, filed on Nov. 1, 2001.
(60) Provisional application No. 60/380,917, filed on May 16, 2002.

(51) Int. Cl. A61B 5/05

(52) U.S. Cl. 600/407

(57) ABSTRACT

This invention utilizes a relatively longer, more penetrating wavelength (compared to the size of the object viewed), but allows more detailed structure to be resolved, by synthetic aperture phased array radar-like detection, which utilizes narrow resonant slits and resonant apertures facing into resonant wave guides and resonant cavities to enhance energy transfer from the electromagnetic radiation source, to form a relatively sharp spatial beam. This beam can be scanned over the sub-scale parts of the object to be viewed. Embodiments of this invention include detector for anthrax) spores in mail envelopes and packages; viewer for brain structure and electrical activity; real-time viewer for stereotactic neurosurgery; detector and destructor of cancer cells and/or cells containing viruses; detector of weapons and explosives.

This application claims the benefit of provisional applications 60/329,785 (filed Oct. 16, 2001); 60/350,454 (filed Oct. 23, 2001); 60/336,076 (filed Nov. 23, 2001); 60/336,389 (filed Nov. 1, 2001) and 60/380,917 (filed May 6, 2002).

### FIELD OF THE INVENTION

This invention relates to detection and identification of microstructures by penetrating electromagnetic radiation of a relatively long wavelength.

This invention relates to detecting microstructure (e.g., anthrax) (bacillus anthracis), and microstructure (e.g., anthrax) spores where such organism may be bare or may be covered by other material, such as an envelope or package.

The technology of this invention also relates to scanning the brain to determine structure and brain electromagnetic activity.

The technology of this invention relates to real-time observation of a stereotactic probe, or other instrument, and a brain or other living tissue in which the stereotactic probe, or other instrument, is inserted in order to visualize and locate the stereotactic probe in real time during neurosurgical, or other, procedures.

This invention also relates to the detection and identification of specific cells, or viruses, such as cancer cells or human immunodeficiency retrovirus; and their destruction.

The technology of this invention also relates to looking through walls to see what objects are in a room, and also relates to the detection and identification of concealed weapons and explosives.

### BACKGROUND

A serious need exists for the fast detection of the presence of microstructure (e.g., anthrax) (bacillus anthraces) spores in mail being sorted by automatic sorting machinery. Such machinery may include a conveyor system for mail and a conveyor system for packages. While biochemical detection methods of the micro-laboratory-on-a chip type are helpful, they still require from an hour to 10 minutes in order to provide a detection alarm. Originally Koch and Pasteur, in the second half of the 18^{th} century studied microstructure (e.g., anthrax) with electromagnetic means, namely light, in the visible wavelength range about 200 nm -800 nm (i.e., 2000-8000 Å). The difficulty with applying these wavelengths is that they only detect the presence of microstructure (e.g., anthrax) spores on the surface of a letter or package. An auxiliary scanning method and apparatus can be set up using appropriate microscopes with associated automatic feature or “target” detection apparatus, set to detect microstructure (e.g., anthrax) spores.

Probing into structures is easier using a relatively longer wavelength. Such electromagnetic radiation (e.g., millimeter or centimeter wavelength microwaves) will penetrate letters and packages.

One difficulty, however, with longer wavelengths is the mismatch with the size of the objects they are trying to detect. The ordinary limit of smallest features detectable by electromagnetic wavelength λ is approximately of the order of that wavelength, λ. The Abbe-Rayleigh theory (Born and Wolf, 2^{nd} edition, p.333 ff, p. 420 ff) expresses the discernable dimension separation between two interfering electromagnetic radiation waves as λ with some additional numerical factors of the order of unity which may depend upon the coherence of the light and on the geometry of the object for which the dimensional resolution is sought.

Brain neuron axons have characteristic diameters of 8 μm to 80 μm. Cancer cells have as lower limit the cells of their normal matrix (e.g., breast tissue). Animal virus dimensions range from poliomyletis (30 nm) to vaccinia (230 nm).

Microstructure (e.g., anthrax) spores may typically have approximate dimensions of a cylinder with a 0.5 μm diameter and a length of 5 μm to 10 μm. It would be desirable to use the millimeter and centimeter microwaves to penetrate envelopes and yet be able to discern the presence of the small microstructure (e.g., anthrax) spores.

The near field effect of using small apertures, i.e., λ>>a, where a is the aperture radius, have been successfully used to increase resolution beyond the Abbe-Rayleigh limit. Ash and Nicholls, for the near field, (Nature, 237, pp. 510-512, 1972) demonstrated a spatial resolution of several millimeters at λ=3 cm using a 1.5 mm diameter circular aperture in a conducting screen. Golosovsky and Davidov, (Appl. Phy Lett., 68 (11), 1996, pp. 1579-1581) also used the near-field for microwave imaging. In contrast to Ash and Nicholls, however, they used a narrow resonant slit (instead of a circular aperture) to achieve a high transmission coefficient (in a limited frequency range) compared to the circular aperture. They were able to get a resolution of 70 μm- to 100 μm at 80 GHz (λ≈3.75 mm). The resolution was therefore about λ/50. Knoll and Keilmann (Nature, 399, pp. 134-137, 1999) used an antenna tip to act as a scattering center. The investigation was done in the infrared and achieved a near-field resolution of 100 nanometers, about λ/100. A scattering tip was actually used in place of an aperture.

Electroencephalography has been used to examine electrical activity in the brain. It has yielded useful results. However, it intrinsically examines an averaged behavior of a very high order of magnitude of neurons.

Typically magnetic resonance images have been used to allow visualization of the structure of a brain before surgery on that brain. The brain may change shape after an incision which affects the pressure of the spinal-cephalic fluid, with other naturally occurring movement of the brain. While a patient may have additional magnetic resonance scanning done during the surgery, the results are not simultaneous with the surgery and often may be difficult to perform during a hiatus in the brain surgery. It is desirable to have a system, method and apparatus which can supply detailed information on the location of a stereotactic probe in real time relative to the actual structure in the brain as visualized in real time.

Various techniques exist for examining a small organism such as a weaponized bacillus or spore, typically with dimensions of the order of 1 micron by 5 micron. These spores may linger in the atmosphere and may be inhaled by humans, resulting in illness and death of the humans. An electron microscope may provide imagery of microstructure (e.g., anthrax) spores. A fair amount of sample preparation for the electron microscope is required. In the current state of world affairs, with microstructure (e.g., anthrax) being sent through the mails and contaminating rooms and personnel, as well as recipients of mail, immediate methods of detection of microstructure (e.g., anthrax) spores is highly desirable.

It is desirable to have a system, method and apparatus which can supply detailed information on the nature and structure of a micro-organism or cellular structure or virus structure identified or visualized in real time.

### SUMMARY OF THE INVENTION

This invention comprises utilization of a relatively longer and relatively more Penetrating wavelength but which allows more detailed structure to be resolved, by its sharpened beam detection, which utilizes relatively narrow resonant slits and relatively small resonant apertures facing into waveguides, or resonant cavities, to ensure a non-degraded energy transfer from the radiation source, e.g. microwave radiation source, to the observable and to the detecting elements., while providing a relatively narrow beam in one or two dimensions from an array of the relatively narrow slits or relatively small apertures.

An embodiment of this invention comprises a method and apparatus for detection of microstructure (e.g., anthrax) spores, for example, on letters and packages in the mail.

An embodiment of this invention comprises a method and apparatus for detecting brain/nerve cell structure and neuron electrical activity in real-time for medical diagnostic purposes as well as research study of the brain and providing for detection of electrical activity on a single neuron or on a neuron bundle. Viewing means comprises holographic as well as other presentations.

An embodiment of this invention comprises a method and apparatus for stereotactic probe imaging such as that of a stereotactic probe in an embedded position in a brain, or spinal column, and showing simultaneously the brain or spinal column microstructures, such as that of a brain (or, spinal column,) of a patient undergoing a neurosurgical procedure, or spinal cord procedure.

An embodiment of this invention comprises a method and apparatus which scans a human body for cancer cells, or cells containing a virus; and detects and destroys cancer cells and cells containing viruses which are susceptible to elevated temperatures.

An embodiments of this invention include a method and apparatus (utilizing a larger scale structure) oriented for detecting features on planets outside our solar system.

An embodiment of this invention comprises utilizes phase differences from the passage of electromagnetic radiation (of the full spectrum used) through the material may provide additional signature information.

An embodiment of this invention comprises a pair of the detectors which may be used in a stereo mode so as to provide a stereo signature.

An embodiment of this invention comprises the scattering of multiple electromagnetic wavelengths provides additional discrimination of the target material from other materials, since the materials have different dielectric constants and complex indices of refraction.

An embodiment of this invention comprises means for additional “target” discrimination utilizes the application of sound waves of an appropriate frequency, while detecting the electromagnetic signature and possible motion of the “target”.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1a shows an exemplary typical resonant circular aperture array layout of an antenna of the microstructure detector;

FIG. 1b shows an exemplary typical resonant slit aperture array layout of an antenna of the microstructure detector;

FIG. 1c shows the experimental layout for testing the theory of a relatively sharp beam;

FIG. 1d shows the layout of a test transmission or aperture grid;

FIG. 1e shows a leftward tilting, beam, but of the order of 2 cm in width;

FIG. 1f shows a rightward tilting beam, but of the order of 2 cm;

FIG. 1g shows a relatively aligned beam of 2 cm cross-dimension;

FIG. 1h shows an upward tilting beam;

FIG. 1i shows an downward tilting beam;

FIG. 2a shows an exemplary configuration of an antenna feed for an element of a resonant slit antenna;

FIG. 2b shows an exemplary configuration of an antenna feed for an element of a resonant slit antenna, able to support a principal mode;

FIG. 2c shows an exemplary configuration of an antenna feed for an element of a resonant annular aperture antenna;

FIG. 2d shows an exemplary configuration of an antenna feed for an element of a resonant annular aperture antenna, able to support a principal mode;

FIG. 2e shows beam-formed propagating electromagnetic waves which progressively scan microstructures, (e.g., microstructure (e.g., anthrax) spores), which are in a package or letter.

FIG. 3 shows a block diagram for the microstructure detector;

FIG. 4a shows a preferred embodiment of the microstructure detector which utilizes resonant slits;

FIG. 4b shows an embodiment of the microstructure detector which operates with a traveling wave in the feed microwave guides, emitting successively at successive slits, as these slits are reached by the traveling wave;

FIG. 5a shows a preferred embodiment of the microstructure detector utilizing resonant slit apertures;

FIG. 5b shows a preferred embodiment of the microstructure (e.g., microstructure (e.g., anthrax)) detector utilizing resonant circular apertures;

FIG. 6a shows the basic mechanism of p-i-n diodes operated by light which results in millimeter/sub-millimeter electromagnetic radiation;

FIG. 6b shows the application of light operated p-i-n diodes to utilize millimeter/sub-millimeter electromagnetic radiation for detection of microstructure (e.g., anthrax) and other microstructures.

FIG. 7a shows the composite visual presentation of one or more microorganism structure and any detectable electrical activity in the microorganisms, including an additional display capability for rotation of a view to display information in different aspects and views;

FIG. 7b shows a letter/package scanned for microstructure (e.g., anthrax) with the probability of microstructure (e.g., anthrax) detected displayed and letter encapsulation and diversion to either observation chamber (to examine microstructure (e.g., anthrax) spores for virulence and for origin) or irradiation chamber (to kill microstructure (e.g., anthrax));

FIG. 8a shows microstructure (e.g., anthrax) detection of a sample collected from air filtration;

FIG. 8b shows microstructure (e.g., anthrax) detector mounted on a mobile military vehicle/civilian vehicle for direct detection of microstructure (e.g., anthrax) spores, or detection using air filtration samples;

FIG. 8c shows microstructure (e.g., anthrax) detector mounted on manned and unmanned aircraft, sea craft, and unmanned missiles for detection of microstructure (e.g., anthrax) or other agents with a known signature;

FIG. 9 shows the stereo version of the “microstructure” or anthrax spore detector;

FIG. 10 shows the brain interrogated by electromagnetic radiation;

FIG. 11a shows the microwave radiation set up to produce a hologram of the brain which is recorded in an electro-active crystal;

FIG. 11b shows a hologram as recorded in an electro-active crystal;

FIG. 11c shows an arrangement for reading out the crystal-recorded microwave hologram by optical region light;

FIG. 12 shows two views of the brain which are separated in time by a small amount being subtracted or added after a first image is delayed in a light-pipe;

FIG. 13 shows a similar addition and subtraction of views of the brain taken at the same time, but at different wavelengths;

FIG. 14 shows a moving antenna element for a synthetic aperture phased-array-like “radar” view of the brain, with a circular and a helical or spiral pathway illustrated;

FIG. 15 shows a fixed array of elements forming a phased array radar, for performing the synthetic aperture-like probing of the brain;

FIG. 16 shows an application of the synthetic aperture-like remote sensing applied to detecting information in a nerve or nerve bundle of the vagus nerve which tells the glucose level to the brain. It is shown with a feedback loop to an insulin pump;

FIG. 17a shows one detector array for both brain and optic nerve;

FIG. 17b shows two phased array detectors, one for arm and hand nerves and the other one for the brain. Not shown is the analysis computer and signal processing elements and chips.

FIG. 18a shows a patient with a stereotactic probe in an embedded position in patient's brain and the phased array imaging transmitter-detector with a schematic representation of an information display;

FIG. 18b shows a synthetic aperture radar, as the electromagnetic transmitter- detector, with a moving antenna placed out of the way of a neurosurgeon as the electromagnetic transmitter-detector;

FIG. 18c shows the composite visual presentation of brain structure and stereotactic probe and the brain electrical activity, shown for selective regions of the brain or for the entire brain, including an additional display capability for rotation of a view to display information in different aspects and views;

FIG. 19 shows interrogating electromagnetic radiation, having spotted a cancer cell, based on its target detection module;

FIG. 20 shows the detector acts to focus many electromagnetic beams 21902 from all angles onto the detected cancer cell(s);

FIG. 21 shows a wearable cancer-detector-eliminator, such as a bra for detecting and preventing breast cancer;

FIG. 22a shows the detector operating at somewhat longer wavelengths used in the field for detecting personnel and material inside a building from the outside;

FIG. 22b shows a stereoscopic version of the detector in FIG. 22a; and

FIG. 23 shows acoustic waves used in conjunction with electromagnetic waves.

### DETAILED DESCRIPTION OF THE BEST MODES

The following description is of the best mode presently contemplated for carrying out the invention. This description is not to be taken in a limiting sense, but is merely made for the purpose of describing the general principles of the invention.

In the following description and in all of the cited figures, no part of a microorganism or human is part of this invention. For example, an microstructure (e.g., anthrax) spore may be depicted by dotted lines, to show generally the relative placement of the parts of the invention. The depicted microorganism is specifically not part of this invention.

### Theory

The general procedure is to detect the structure of the microorganism in question with a synthetic aperture radar-like apparatus, in some form. Other signature information of the microorganism may be available from the electromagnetic return signal. An apparatus may be readily constructed because much of the needed components are known

In order to overcome the apparent diffraction limitations on far field electromagnetic field resolution one may start with the observation that ordinary radar beams are diffraction limited to a linear resolution of λ R/D where λ is the wavelength of operation of the radar, R is the range at which detection occurs, and D is the horizontal aperture of the antenna. On the other hand, unfocused simulated aperture radar (SAR) has a resolution of ½ (λR)^{1/2} while for the focused SAR cases the resolution is D/2 (cf. Skolnick, ed., Radar Handbook 2^{nd} Ed., McGraw Hill, New York, p. 21.4). The difference between the focused and the unfocused SAR is that the focused SAR provides for phase adjustment of the received signals, while the unfocused SAR does not do phase adjustment before the received coherent signals are integrated.

The assumptions involved in the above derivation of resolution for the focused SAR include the assumption that the wavelength, λ, is much smaller than the aperture (classical diffraction conditions), λ<<D. Consequently, it may be desirable to try to use a very small aperture, for example, 1 nm to 1 μm, so as to achieve a resolution of the examined object of that order of magnitude, namely, 1 nm to 1 μm.

In the very small aperture case, D<<λ, however the formula for the focused SAR resolution, i.e., D/2, may no longer apply since it was derived under the assumption λ<<D. Therefore one must look at the case with D<<λ carefully to see what the resolution may actually be. Bethe solved the problem of electromagnetic transmission through an aperture for the case where D<<λ (1944, Physical Review, (2) 66, “Theory of Diffraction by Small Holes”). One may use the electromagnetic form of Babinet's principle, interchanging small object with a small aperture to guess the solution (Born and Wolf, 1962, Principles of Optics, 2^{nd} Edition, pp. 559-60). As Bethe (1944) showed, all cross sections correspond to Rayleigh's theory of scattering by small objects (Born and Wolf, p. 649 ff). The aperture cross section, setting the radius of the aperture a=D/2, is also proportional to (a)⁶. The cross section corresponds to the scattering of a dielectric sphere or a disk of radius a and a dielectric constant of 2.

Bethe calculated the near and far electromagnetic field. Bouwkamp (Phillips Research Reports 5, 321-332, 1950) derived a near field modification to Bethe's calculation. Bethe's far field electromagnetic equations were unaffected. At larger distances (>>λ), the far field electric and magnetic vector fields are:

E=1/(3π)k²(a)³φ₀κ×(2H₀+E₀×κ)

and

H=−1/(3π)k²(a)³φ₀κ×(2H₀×κ−E₀)

where k λ=2π, a is the radius of circular aperture, φ₀=e^{ikr}/r, κ is the direction of propagation of the diffracted electromagnetic wave, H₀ and E₀ are the magnetic and electric fields on the incident side of the conducting screen as though there were no hole in the conducting material. The symbol×represents the ordinary vector cross-product.

The Poynting vector (S), i.e., the energy flux, of the diffracted field is (Bethe, 1944)

S=c/4πE×H=(c/36π³)(k⁴a⁶/r²)κ(2κ×H₀−κ×κ×E₀)2

The intensity of radiation in the direction of the electromagnetic wave propagation is, per unit solid angle,

R²|S|=(c/36π³)k⁴a⁶[4H₀² cos² θ cos² α+(sin θE_{0,x}−2H₀ sin α)²].

The radiation is not symmetrical about any axis. The angle θ is the angle between the propagation of the electromagnetic wave κ and the normal to the conducting screen. The angle a is the azimuth angle of κ as it is rotated from H₀.

The energy flux integrated over all angles is given by

$${S}_{tot} = {\int }_{0}^{2 π} sin θ . ⅆ θ {\int }_{0}^{2 π} ⅆ α . {r}^{2} \vert S \vert = c / ( 27 {π}^{2} ) {k}^{2} {a}^{6} ( 4 {H}_{0}^{2} + {E}_{0}^{2} )$$

where E₀² and H₀² time averages of these fields.

Bethe's diffraction solution (λ>>a) has

H_{Bethe}≈k²a³H₀

whereas for λ<<a (Kirchhoff)

H_{Kirchhoff}≈ka²H₀

For small holes, λ>>a, the radiation transmitted through the hole is much smaller than for the Kirchhoff theory. The E and H fields are reduced by a factor of ≈a/λ. The power is reduced by a factor of (a/λ)².

The cross-sections are proportional to λ⁻⁴. This is the same as in Rayleigh's theory of scattering for small particles (a<<λ). The proportionality of the small aperture cross-section (≈a⁶) also corresponds to the Rayleigh small particle scattering cross-section of a dielectric sphere or disk of radius a and dielectric constant of about 2. (See also R. Collin, Field Theory of Waveguides, McGraw Hill, New York, 1960, chap. 7; R. Collin, Foundations for Microwave Engineering, McGraw Hill, New York, 1992, p.181ff.).

### Approximation

For a monochromatic scalar wave V(x,y,z,t)=U(x,y,z)e^{-iωt} and the spatial dependent part satisfies the time independent wave equation (∇²+k² )·U=0. The idea of Huygens and Fresnel is that the electromagnetic disturbance at a point P arises from the superposition of secondary waves that proceed from a surface situated between this point and the electromagnetic source. Taking the elementary spherical wave solution for a Green's function, the solution for a point P may be written as (2):

$$U ( P ) = \frac{1}{4 π} \int {\int }_{S} \{ U \frac{\partial }{\partial n} ( \frac{1}{s} ) - \frac{1}{s} \frac{\partial U}{\partial n} \} ⅆ S$$

No assumption has been made as to the relative size of wavelength and aperture. Levine and Schwinger (Levine and Schwinger “On the Theory of Diffraction by an Aperture in an Infinite Plane Screen.” Phy. Rev. 74, 958, (1948) )solve the time independent wave equation (∇²+k²)·U=0. The solution in their notation is:

$$\begin{matrix}φ ( r ) = 2 i sin ( kz cos {ϑ}^{!} ) {exp}^{( ⅈ {kn}^{′} ρ )} + {{\int \int }_{{S}_{1} {k}_{x} \cdot k , = ∞}}^{∞} {φ ( {r}^{′} )}_{{z}^{′} = 0} \frac{\partial }{\partial {z}^{′}} {\{ A \}}_{{z}^{′} = 0} ⅆ {k}_{x} ⅆ {k}_{y} ⅆ {S}^{′} \\ where {\{ A \}}_{{z}^{′} = 0} = \{ \frac{exp [ ⅈ ⟨ {k}_{{k}_{x}} ( x - {x}^{′} ) + {k}_{y} ( y - {y}^{′} ) + ( {k}^{2} - {k}_{x}^{2} - {k}_{y}^{2} ) \frac{1}{2} ( {z}^{′} - z ) ⟩ ]}{4 {π}^{2} i ( {k}^{2} - {k}_{x}^{2} - {k}_{y}^{2} ) \frac{1}{2}} \}\end{matrix}$$

Therefore one estimate that the far electromagnetic field can be approximated by the spatial Fourier transform of the source. For example, where there is a regular array, and to first order the sources are at each array aperture or array slot, the Fourier transform looks like sin (½NdZ)/N sin(½dZ) The quantity N is the number of array apertures, d is the (uniform spacing) between apertures, and Z=cos θ−cos θ₀.

The angle θ₀ the scan angle and the angle θ is the angle for the field is calculated. A one dimensional array is assumed.

In this invention, small apertures are used in a phased array mode. The configuration may be mono-static or bi-static. One may characterize the basic invention as a “phased array synthetic aperture radar system.”

The half-power at half-height (−3db) beam width angle θ₃ of a phased array antenna may be written in terms of the product of Nd, where N is the number of elements for a large array and d is the spacing between elements. Utilizing Nd in place of a as a measure of the aperture relating to the beam width angle for large N, one may solve (Hansen, 1998, Phased Array Antennas, John Wiley & Sons, New York, pp. 9-1 0)

$$sin \frac{1}{2} {Nkdu}_{3} / ( N sin \frac{1}{2} {kdu}_{3} ) = \sqrt{0.5}$$

for the half power points for a large array (N>>1) which yields

$$\frac{1}{2} {Nkdu}_{3} = \pm 0.4429$$

For a beam scanned at angle θ₀, the 3 dB beam width (θ₃) is

$${θ}_{3} = {sin}^{- 1} ( sin {θ}_{0} + 0.4429 \frac{λ}{Nd} ) - {sin}^{- 1} ( sin {θ}_{0} - 0.4429 \frac{λ}{Nd} )$$

which, for large N, reduces to

$${θ}_{3} ≅ \frac{0.8858 λ}{Nd cos {θ}_{0}}$$

Then, following the typical derivation of azimuth resolution (δ), for focused SAR

$${β}_{eff} = \frac{λ}{2 {L}_{eff}}$$

$$Leff = R {θ}_{3} ≅ \frac{0.8858 R λ}{Nd cos {θ}_{0}}$$

A phased array angular beam spread is at a relatively small angle,

δ=β_{eff}R

where R is the range and β_{eff} is the effective half-power beam width Combining, β_{eff}, L_{eff} and δ:

$$δ = \frac{λ}{2 {L}_{eff}} R = \frac{λ \cdot RNd cos {θ}_{0}}{2 \cdot 0.8858 R λ} = \frac{Nd cos {θ}_{0}}{1.7716}$$

Consequently, the resolution depends on the number of array elements (N) and their spacing (d). Of course θ₀ also affects the resolution. There is also an array element beam factor (not shown) which incorporates the effects of small aperture as well as element beam shape.

### Feasibility

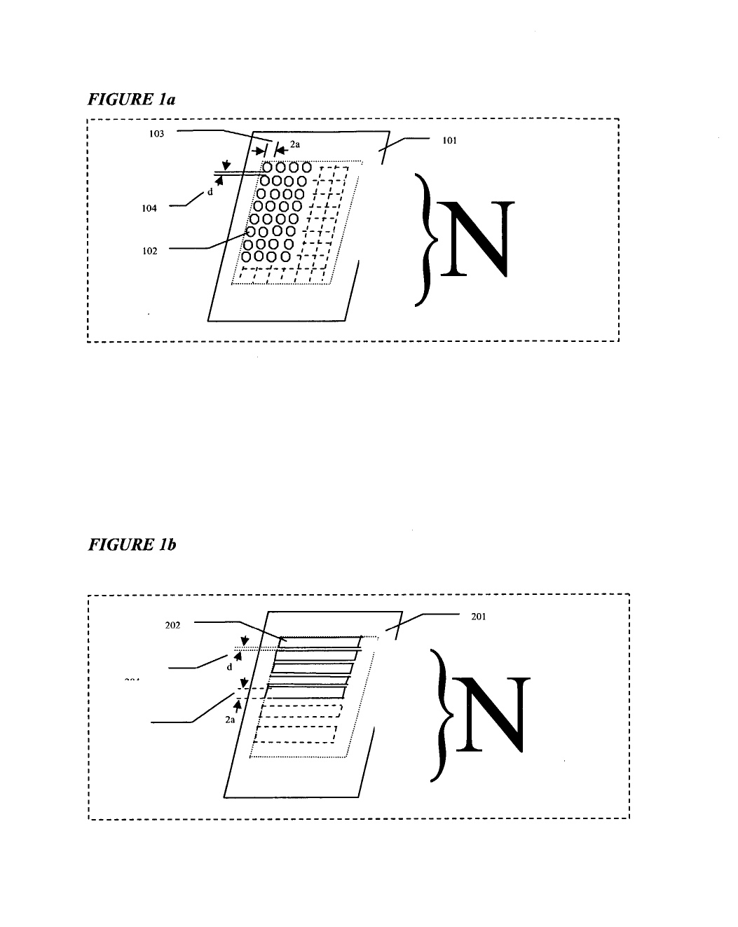

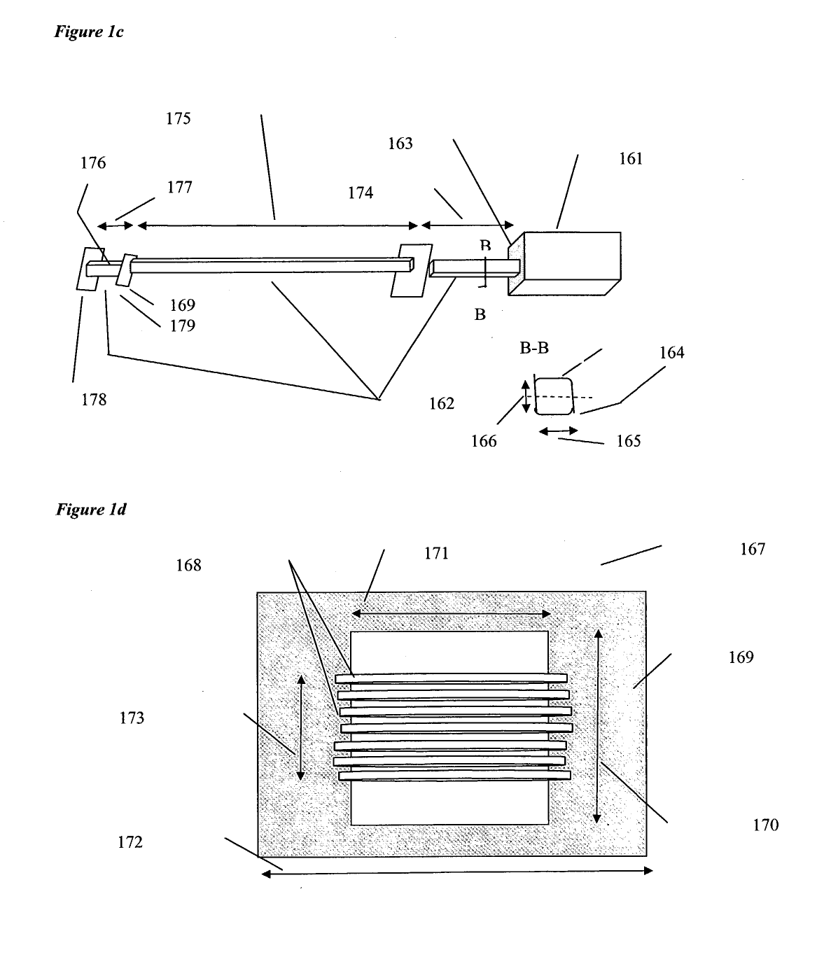

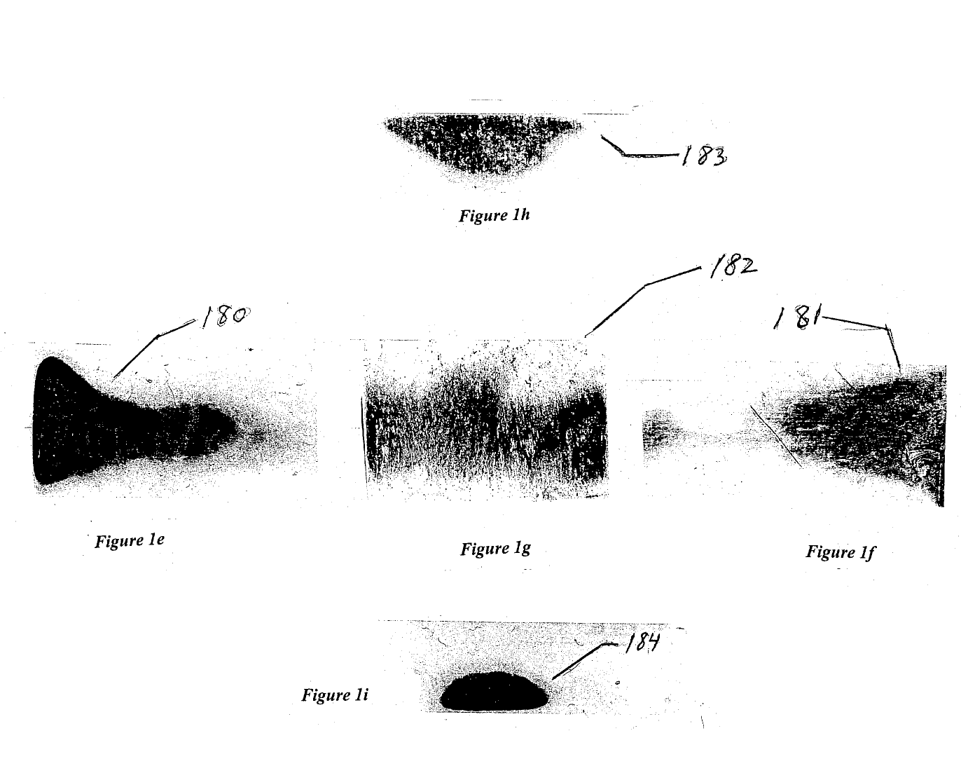

In order to get the most power through the aperture, the aperture should be made as large as possible. A typical array layout may be as shown in FIG. 1a. FIG. 1a shows an exemplary typical resonant circular aperture array layout of an antenna of the microstructure (e.g., microstructure (e.g., anthrax)) detector. A conducting sheet 101 has circular apertures 102. The diameter of the circular apertures 102 is 2a 103. The spacing between the circular apertures 102 is the distance d 104. The spacing of the elements d and their total number N may be such that Nd≅100 nm (100×10⁻⁹ m).

FIG. 1b shows a similar array, but utilizes thin slit apertures, instead of circular apertures, in order to achieve a more efficient coupling to the aperture (cf. Golosovsky and Davidov, Appl. Phy. Lett., 68 (11), 1996, pp. 1579-1581). FIG. 1b shows an exemplary typical resonant slit aperture array layout of an antenna of the microstructure (e.g., microstructure (e.g., anthrax)) detector. Here, a conducting sheet 201 has elongated slits 202. The width of the slits is 2a 203. The separation of the slits is d 204.

Examining the radar equation which gives the received signal power Pᵣ as

$${P}_{r} = \frac{{P}_{t} {G}_{t} {A}_{e} σ}{{( 4 π )}^{2} {R}^{4}}$$

where Pₜ is the transmitter power which has a gain of Gₜ. R is the one way range. The cross section σ is in square meters. The receiving antenna has an effective aperture of Aₑ.

In order to estimate the feasibility of the power requirements for a far-field observation of objects of size of microstructure (e.g., microstructure (e.g., anthrax)) spores (approximate dimensions of a cylinder with a 0.5 μm diameter and a length of 5 μm to 10 μm) we utilize the known parameters of the Canadian satellite RADARSAT-1. This satellite carries a simulated aperture radar. The relevant satellite parameters are available and can be related to the far-field parameters for the microstructure (e.g., microstructure (e.g., anthrax)) spore observations.

The RADARSAT-1 has an orbit 783 km above the surface of the earth. It's antenna is 15 m by 1.5 m. The peak transmitter power is 5 kW while the average power is 300 W. The wavelength is 5.6 cm (5.3 GHz, C-band).

The radiation intensity from the RADARSAT-1 is reflected off the ground surface and is usefully detected by the on-board radar receivers. Therefore, comparing the radar equation for the received power for an microstructure (e.g., microstructure (e.g., anthrax)) detector (superscript/subscript label A) with that of the RADARSAT-1 (superscript/subscript label S) allows for an estimate of the microstructure (e.g., microstructure (e.g., anthrax)) detector receiver power requirements.

Pᵣ^{S}/Pᵣ^{A}=Pₜ^{S}Gₜ^{S}Aₑ^{S}σ^{S}R_{S}⁻⁴/Pₜ^{A}Gₜ^{A}Aₑ^{A}σ^{A}R_{A}⁻⁴

where

ƒ₁=Pₜ^{S}Gₜ^{S}/Pₜ^{A}Gₜ^{A}=1 kW·a_{A}⁻⁶a_{S}⁴·λ_{S} ⁻²λ_{A}⁴

and

ƒ₂=R_{S}⁻⁴R_{A}⁴

substituting numerical values from above for the respective elements of satellite (S) and microstructure (e.g., microstructure (e.g., anthrax)) (A) detectors, remembering that the microstructure (e.g., microstructure (e.g., anthrax)) detector range is order of 1 cm (=10⁻2 m).

Then

ƒ₁=(1 kW)·(10⁻⁷)⁻⁶(10)⁴(5.5·10⁻²)⁻²(5.5·10⁻³)⁴=3.0·10³⁸

ƒ₂=(10⁶)⁻⁴(10⁻²)⁴=10⁻³²

Therefore

Pᵣ^{S}/Pᵣ^{A}∝ƒ₁·ƒ₂=3·10⁶

One may conclude that the microstructure (e.g., anthrax) detector detection is feasible, but one may have to work a little harder for better receiver sensitivity (additional sensitivity of 10⁻³ to 10⁻⁶) and one must keep the signal to noise ration high (by a large number of “hits”); and one might increase the radar transmitter output power for the microstructure (e.g., anthrax)) detector radar transmitter to megawatts thereby gaining a factor of 10⁻³.

The scattering cross section of the microstructure (e.g., anthrax)), however, will be proportional to a⁶, where a is the order of 1 μm (10⁻⁶ m), there would otherwise be an additional factor of (10⁻⁶)⁶=10⁻³⁶ which would be difficult to overcome.

The conclusion is to resort to resonant slots and apertures while maintaining the microstructure (e.g., anthrax) detection apparatus chamber as part of a waveguide or as part of a resonant cavity. It is known that for various slit dimensional relationships to the waveguide dimensions, resonance may be found such that the losses going through a slit may be kept to a loss of around 33% of the incident power incident on the other side of the slit, Felsen, L. B. and N. Marcuvitz, Slot Coupling and Spherical Waveguides, J. Appl. Phy., 24, no. 6, (1953), pp. 755-770. The wavelength used was 3.2 cm (λ_{free space}) with slots in a waveguide of inside dimensions, a=2.28 cm and b=1.01 cm. Slot sizes included a′=1.59 cm and b′=0.4 cm, wherefore the slit width b′ is thus 0.12 λ. (Also see: H. Bethe, op. cit., pp.178-182; and T. Mareno, Microwave Transmission Design Data, Dover, New York, 1958, pp. 210-241).

### Experiment

Golosovsky and Davidov have reported (Applied Physics Letters 68 (11), 11 Mar. 1996. pp.1579-81) the use of a resonant slit for near-field sub-wavelength apertures. Their particular application is for a near-field resistivity microscope. They achieved their required effect by mounting a thin conducting sheet across a rectangular waveguide with dimensions a and b.

Corresponding to the dimensions a and b, there may be a narrow slit with width b′ and length a′. The condition for resonance or “transparency” of the slit to the microwave radiation is (T. Moreno, op. cit. p. 154)

$$\frac{a}{b} \sqrt{1 - {( \frac{λ}{2 a} )}^{2}} = \frac{{a}^{′}}{{b}^{′}} \sqrt{1 - {( \frac{λ}{2 {a}^{′}} )}^{2}}$$

When b′ goes to zero, the equation above shows a corresponding limit

λ=2a′−(b′/b)²(a²/a′−a′)

which indicates that very narrow slits are resonant at some wavelength, which can be controlled by the choice of a′ and b′.

Moreno (op. cit., pp. 156-7) shows some aperture structures in circular waveguides carrying only the dominant mode. These include annular and semi-annular apertures. It is also known that waveguides which are not simply connected contain the principle mode (Landau and Lifschitz, Continuous Fields in Inhomogeneous Media) which is not subject to a cutoff wavelength, where such cutoff wavelengths produce effervescent non-propagating waves. Therefore the annular resonant aperture may be used across an annular or coaxial waveguide or resonant chamber.

J. C. Slater analyzed transfer of power through resonant apertures based on impedance matching (Microwave Transmission; 2^{nd} edition, Dover Publications, New York, N.Y., 1959; original copyright 1942). Slater considers a rectangular waveguide with a larger cross-section dimension “a” and a smaller cross-section dimension “b”. He defines an equivalent impedance, Z. For the TE₁₀ mode of a rectangular waveguide, the current (i) flowing is the magnetic field H times the distance a while the voltage (V) is the electric field E times the distance b. The impedance Z is then V/i:

Z=V/I=E/Hb/a=(μ/ε)^{1/2}1/[1−(λ₀/2a)²]b/a

where Z=E/H=(μ/ε)^{1/2}1/[1−(λ₀/2a)²] was derived from waves in rectangular waveguides which vary exponentially along the z-axis and sinusoidally along the x- and y-axes.

Rewriting,

b²=ε/μZ2[a²−(λ/2)²]

which shows that a and b are on a hyperbola, if Z is held constant. Therefore any two pairs of points lying on such a hyperbola; a represent waveguides whose impedances match.

An experiment was performed to produce a beam of about 1 cm or 2 cm width. The choice of dimensions was based on Moreno (op. cit.) and Slater (op. cit). A microwave oven 161 operating at 2.45 GHz was modified (See FIG. 1c) to accommodate a waveguide 162. A rectangular hole 163 was drilled and sawed into the side of the microwave oven away from the entrance of the microwaves into the oven.

The waveguide 162 was made from two sections of steel channel 164. The sections were taped together with metallic duct tape so as to have dimensions 3.63 inches (9.22 cm) 165 by 2.00 inches (5.08 cm) 166, as can be seen in cross-section B-B. The aperture or transmitter grid 167 (FIG. 1d) was constructed from copper wire 168 soldered to a steel plate 169 with an opening 1 15/16 inches (4.92 cm) 170 by 3 inches (7.62 cm) 171. The outside of the plate was 4 1/16 inches (10.32 cm) square 172, being a cover for a steel electrical outlet box. The wires 168 were 0.051 inches (1.30 mm). The wires were about 1.30 mm apart. The total width 173 of the aperture or transmitter grid 167 was 2 cm. Any open space outside of the area of the aperture or transmitter grid 167 was covered with heavy duty aluminum foil to block spurious microwave transmission.

The aperture or transmitter grid was mounted 24¼ inches (61.60 cm) 173 from the magnetron, down the wave guide. The detector for the scattered radiation was a further 86¼ inches (219.08 cm) 175 down the waveguide. The termination section 176 was 9 inches (22.86 cm) long 177 and was closed at the far end with a steel plate 178. The entrance to the termination section held a steel plate 169 like the plate to which the aperture grid wires were soldered. This steel plate 169 held the detector material 179.

The detector material 179 was thermal fax paper which had been soaked in water for a few minutes and then blotted dry on the surface. The wetted thermal fax paper operated to absorb the microwaves and become hot enough to darken in places where the electromagnetic radiation was strongest. The wetted thermal paper was an integrating measurer. The other control available was the time during which the microwaves were being continuously generated by the magnetron.

The results tend to prove the theory. FIG. 1e shows a rightward tilting, beam 180, but of the order of 2 cm in width. FIG. 1f shows a leftward tilting beam 181, but of the order of 2 cm. FIG. 1g shows a relatively aligned beam 182 of 2 cm cross-dimension. FIG. 1h shows an upward tilting beam 183. FIG. 1i shows a downward tilting beam 184.

The basic radiation wavelength from the microwave oven is 12.24 cm (4.82 inches). In the far field, at a distance of 219.1 cm, which is 17.9 times the wavelength (12.24 cm) and 28.8 times the long dimension of the slit (3 inches, 7.62 cm) and 1685.4 times the slit separation (1.30 mm) and the distance between the slits (1.30 mm), the results indicated a sharpened beam was formed of the correct dimensions., about 2 cm, while the wavelength used was about 12.24 cm.

### Detector

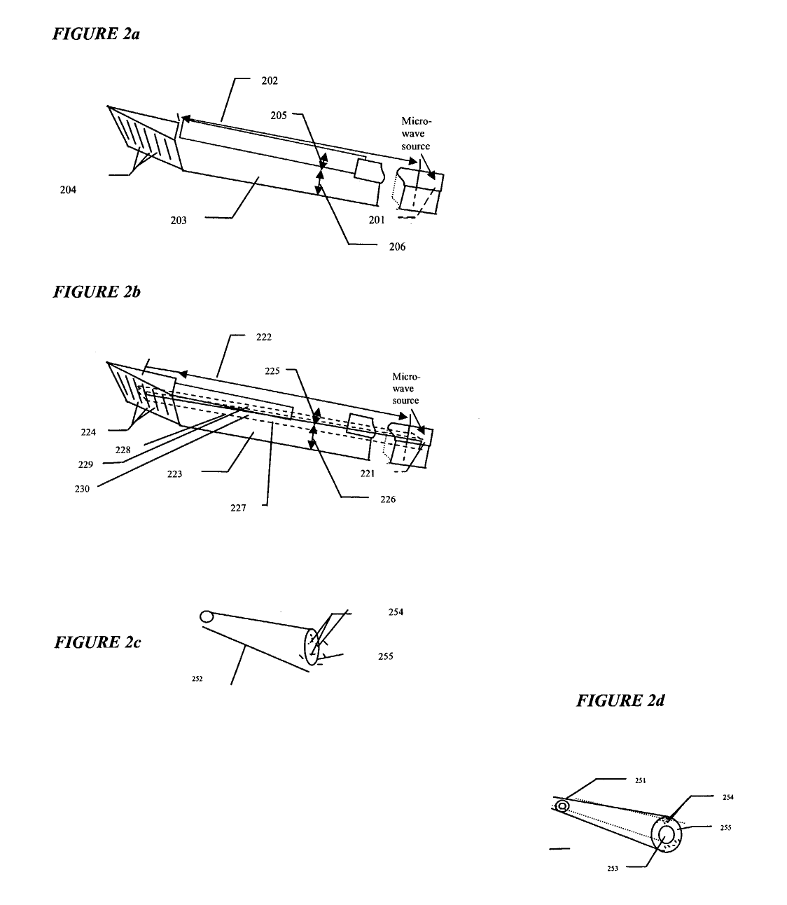

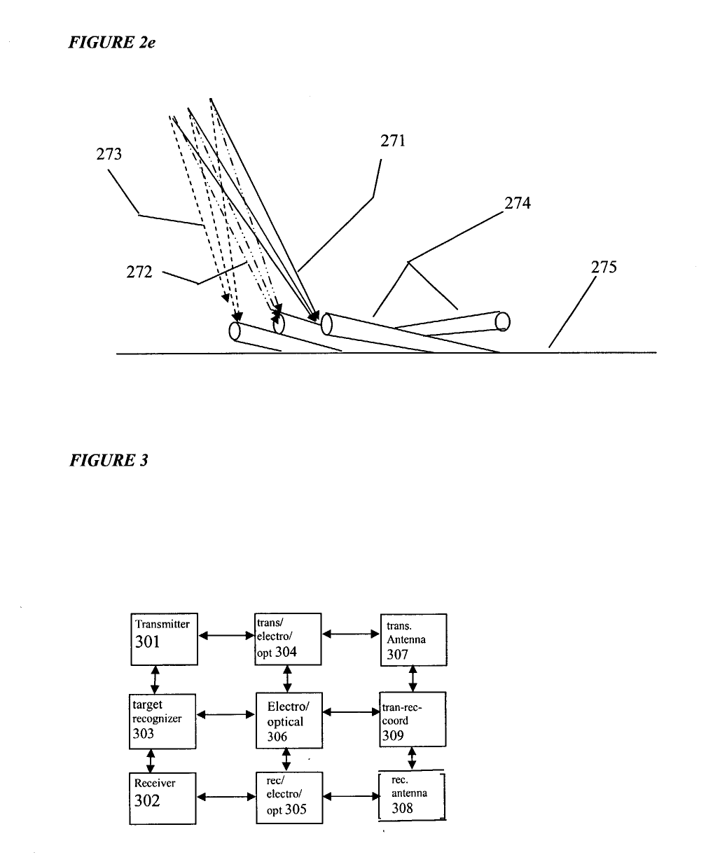

FIG. 2a shows an exemplary configuration of an antenna feed for an element of a resonant slit antenna. The microwave source 201 propagates an electromagnetic wave down the rectangular waveguide 203 which has dimensions of length l 202, width w 206 and height h 205. The antenna-like end 204 of the microwave guide 203 is shown with elongated slit apertures 204.

FIG. 2b shows an exemplary configuration of an antenna feed for an element of a resonant slit antenna, able to support a principal mode. The microwave source 221 propagates an electromagnetic wave down the rectangular waveguide 223 which has dimensions of length l 222, width w 226 and height h 225. The antenna-like end 224 of the microwave guide 223 is shown with elongated slit apertures 224. In addition, an inner conducting structure 227 of rectangular cross section forms a non-simply connected waveguide which can therefore support a principle mode and is not subject to a cut-off wavelength. The length l ′ 228 of the inner structure is equal to the length l 222 of the outer waveguide. The width w′ 229 of the inner structure is less than the width w 226 of the outer waveguide and the height h′ 230 of the inner structure is less than the height h 225 of the outer waveguide.

FIG. 2c shows an exemplary configuration of an antenna feed for an element of an annular aperture antenna. The waveguide feed is a circular cross section conducting tube 251 with a transverse conducting sheet 255. The transverse conducting sheet 255 has circular apertures 254, only a few of which are indicated. For the resonant circular apertures, the arrangement of the aperture geometry is more straightforward than for the slit case, since there is intrinsic aperture symmetry in two dimensions.

FIG. 2d shows an exemplary configuration of an antenna feed for an element of an annular aperture antenna, able to support a principal mode. The waveguide feed is a circular cross section conducting tube 251 with a transverse conducting sheet 255. The transverse conducting sheet 255 has circular apertures 254, only a few of which are indicated. In addition, an inner conducting cylinder 252 which has a radial dimension less than that of the outer cylindrical waveguide 251, but is of equal length. A portion 253 of the transverse conducting plate 255 does not contain any circular apertures 254. That portion 253 corresponds to the cross section of the inner conducting cylinder 252. The inner cylinder creates a non-simply connected waveguide which may support a principle mode which is not subject to a cutoff wavelength.

The consequences of a resonant slit or aperture (coupling resonant cavities) for the transmitting and receiving structures is that the Rayleigh-type cross section for the microwaves (λ>>a ) interacting with the small target (σ≈100 nm, i.e., 10010⁻⁹) ends up being detectable from a power budget point of view.

FIG. 2e shows a beam-formed propagating electromagnetic waves 271 which have progressively scanned 272, 273 microstructures (e.g., anthrax) spores) 274 which are sitting on the bottom of an envelope 275. For example, the features of 100 nm, equivalently, 0.1 μm, are discemable, under the corresponding choice of a wavelength of 0.5 mm with slit (or aperture) dimensions of 1 nm width (or diameter) with a spacing of 1 nm, where the slit (aperture) elements are activated as a phased array with 100 slits (apertures) in a beam-forming group. These numbers or quantities are idealized. In a constructed operational radar system, engineering tolerances may contribute variations to these numbers.

One might use a synthetic aperture radar, with a moving antenna, or as an equivalent, a fixed phased array radar where the width of the physical antenna D is chosen so that the simulated aperture radar resolution D/2 is such as to provide a desired resolution of the micro-organism scrutinized. Although the wavelength, typically millimeter waves, are longer than the dimension of the target, for bacillus anthraces spores about 5 μm, the “spotlighting” by the phased array radar (operating in a simulated aperture mode) provides orders of magnitudes more “hits” on the target, to counteract the lesser interaction Hamiltonian of the longer waves.

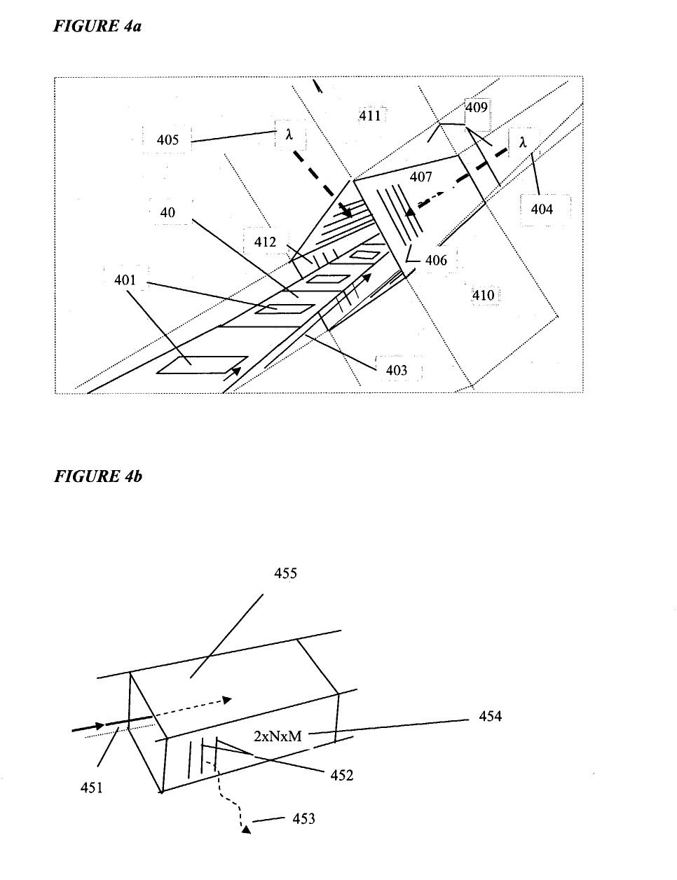

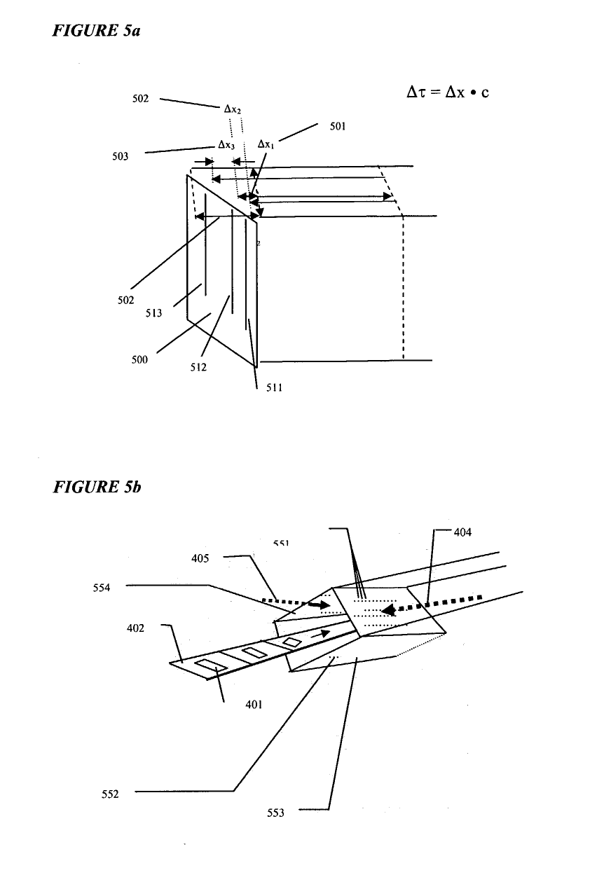

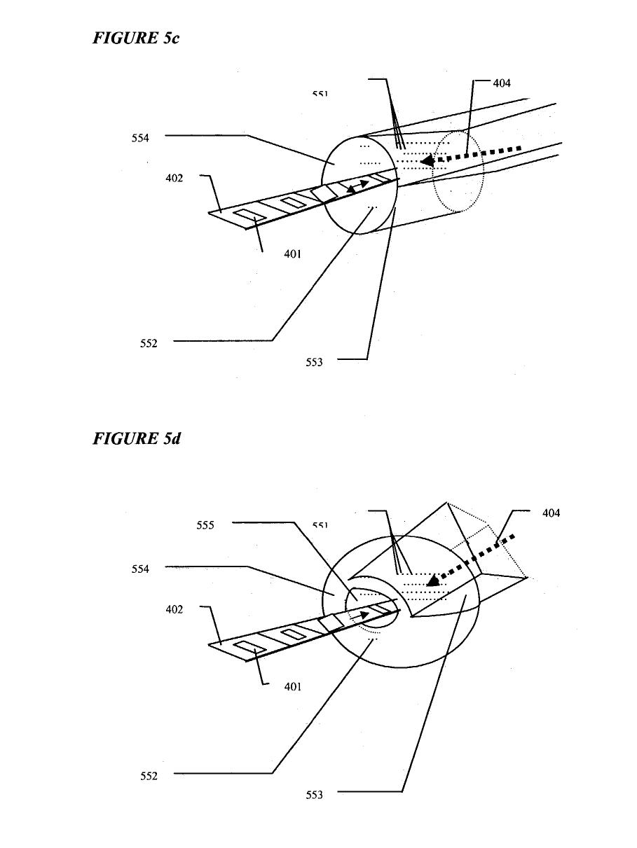

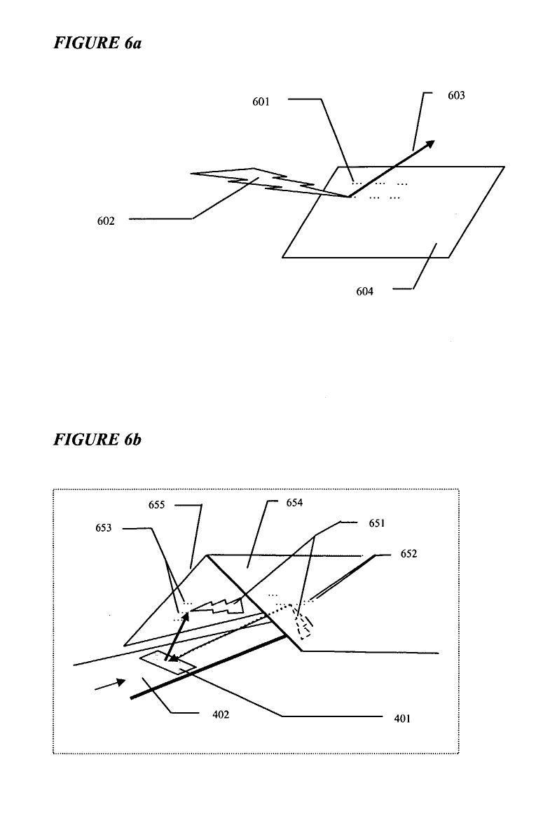

FIG. 3 shows a block diagram for the microstructure (e.g., anthrax) detector. The block diagram shows a transmitter section 301 and a receiver section 302. These sections perform the traditional radar functions including all processing and associated processing, including software and hardware methods. A target recognizer 303 utilizes all available information required for it to define and recognize a target, among a set of targets incorporated into a memory function of the target recognizer. The target recognizer may utilize the nanometer scale structural details of which the radar is capable. These nanometer scale details, as described so far, particularly relate to the “cross range” direction. However, as will become apparent below (FIGS. 4a, 5b) the cross-range may be simultaneously determined in an x and y direction, relative to the ranging direction z, in a Cartesian coordinate system. As will also become apparent, (FIGS. 6a and 6b) utilizing terahertz microwave bursts, which may be additionally chirped, will discriminate highly detailed structure in the ranging direction (z) so as to add to the ability of the target recognizer to successfully identify microstructures.

The block diagram (FIG. 3) also shows a transmitter electro-optical section 304 and a receiver electro-optical section 305. For a conventional radar setup only, sections 304, 305, 306 and 309 are electronic functions only or electronic and conventional optical functions only. However, as will be show below, utilizing terahertz radiation pulse and radiation inducing techniques, additional ranging detail capability may be added. The electro-optical section 306 includes all central electro-optical functions not included in the specific transmitter electro-optical functions 304 and receiver electro-optical functions 305. The transmitter antenna functions 307 and the receiver antenna functions 308 may be either “conventional” resonant slit or aperture antennae, or, for example, “non-conventional” p-i-n diode antennae. The transmit-receiver coordination function 309 may include utilizing light pulses to induce a p-i-n diode array antenna to emit terahertz radiation of the required wavelength (e.g., 0.5 millimeter=5×10⁻⁴ m.). At the same time a delayed form of the light may be utilized to gate a receiver. This process may act to increase the sensitivity of the radar system and achieve a better ability to detect a signal in the presence of noise.

FIG. 4a shows a preferred embodiment of the microstructure (e.g., anthrax) detector which utilizes resonant slits. This embodiment of the microstructure (e.g., anthrax) detector may operate with a traveling wave in the feed waveguides, emitting successively at successive slits, as these slits are reached by the traveling wave. In a preferred embodiment as shown in FIG. 4a, letter or package mail 401 is carried along on a conveyor belt 402, in the direction shown by the arrow 403. Two propagating microwave radiation streams are shown, 404 and 405. The resonant antenna slits 406 are shown in an orientation on a conducting transverse sheet 407. The propagating electromagnetic radiation in another stream 405 is shown impinging on resonant antenna slits in an orthogonal direction to the slits 406 which serve the propagating electromagnetic microwave stream 404. The orthogonality of the resonant antenna slits allows for “cross-range” details in two directions, albeit from two differing viewing angles. This may provide for a stereoscopic view. The receiving and target recognizer functions may contain subsections to handle the assembly of these data.

It is important to understand that the incoming propagating electromagnetic radiation enters from a waveguide 411 through “transmitter” slits (or apertures) in a conducting sheet and scatters off of the material inside a mail envelope as well as the envelope, for example. The scattered propagating electromagnetic radiation exits through “receiver” slits (or apertures) 412 in a conducting sheet covered with “receiver” slits (or apertures) 412 located on an opposite waveguide or other structure. The received scattered radiation (including forward scattered radiation) is received by and analyzed by the radar receiver functions (302, 305, 308, FIG. 3). Additionally, the target recognizer radar function (303, FIG. 3) analyzes the received and analyzed radar data to determine if, for example, if any microstructure (e.g., anthrax spores are present.

The transmitted incoming beam is sharpened as it passes through the “transmitter” slits (or apertures). Further, by the action of time delays, or other passive or active artifices or means known in the arts, the transmitted beam is swept across its target, scattering, with target information, from small features of the target, of the order of Nd, where N is the number of slits (or apertures) forming the beam at a given time and d is their separation.

The propagating electromagnetic radiation stream 404 enters from the diagonal from the upper right. Three of the walls of the rectangular waveguide are denoted 409. The propagating electromagnetic radiation stream 405 enters from the diagonal from the upper left. The entering waveguide 411 may be followed by an exiting waveguide. The configuration as shown is bi-static with separated transmitters and receivers. It could also be configured in a monostatic system.

A different embodiment, which is similar to the embodiment as shown in FIG. 4a, with the difference that no opposite (or exiting) waveguides are present. This embodiment is not shown.

FIG. 4b shows such an embodiment of the microstructure (e.g., anthrax) detector which operates with a traveling wave in the feed microwave guides, emitting successively at successive slits, as these slits are reached by the traveling wave. The propagating electromagnetic wave 451 propagates down the waveguide 455 where resonant slits 452 act as antenna elements with resultant “transmitted” propagating electromagnetic radiation 453. Only a few of the resonant slits are depicted, and these are depicted in a “macro” fashion, for didactic purposes. The actual slit widths may be 1 nanometer (1×10⁻⁹ m) wide. The slits may be separated by 1 nanometer (d). There may be successive groups of 100 slits (N). There may be 5×10⁶ slit groups (M). Thus, where the slit groups are separated by 1 nanometer, the total distance covered by the slits may be one meter (2×N×M 454). For every application of the detection system, the actual number of slits required may be different.

FIG. 5a shows a preferred embodiment of the microstructure (e.g., anthrax) detector utilizing resonant slit apertures. A transverse conducting sheet 500 is tilted diagonally so that an additional distance Δx presents itself for a propagating microwave to traverse, Δτ=Δx·c, where Δτ is the addition time to traverse an additional distance Δx and c is the speed of the wave propagation. The sub-distances Δx₁ 501, Δx₂ 502 and Δx₃ 503 corresponds to different resonant slits 511, 512, 513 such that a phased delay wave front is propagated out of the plane of the figure.

FIG. 5b shows a preferred embodiment of the microstructure (e.g., anthrax) detector utilizing circular apertures which feed into a resonant cavity. The general layout is similar to a preferred embodiment shown in FIG. 4a. In the present preferred embodiment, as shown in FIG. 5b, letter or package mail 401 is carried along on a conveyor belt 402. Two propagating microwave radiation streams are shown, 404 and 405. Only a few of the circular apertures 551 are shown. These circular apertures 551 are the order of nanometers (1×10⁻⁹ m.), separated by distances of the order of nanometers. Receiving circular apertures 552 are shown on a conducting sheet 553, for the second propagating microwave radiation stream 405. Receiving circular apertures (not shown) are also present on the analogous conducting sheet for the first propagating microwave radiation stream 404. Embodiments of the structure of the radar may utilize nanotechnology. Holes or slits (narrow dimension) of the order of 1 nm (1×10⁻⁹ m) may be formed in the walls of waveguides, with spacing of the order on 1 nm, all in relatively regular placement (alignment to within a fraction of 1 nm). Otherwise, the embodiment shown in FIG. 5b is analogous to the embodiment shown in FIG. 4a with entrance and exit waveguides for each propagating microwave radiation streams 404, 405.

FIG. 5c shows a preferred embodiment of the microstructure (e.g., anthrax) detector utilizing circular apertures facing into a resonant cavity. The general layout is similar to a preferred embodiment shown in FIG. 4a. In the present preferred embodiment, as shown in FIG. 5c, letter or package mail 401 is carried along on a conveyor belt 402. A propagating microwave radiation stream 404 is shown. Only a few of the circular apertures 551 are shown. These circular apertures 551 are the order of nanometers (1×10⁻⁹ m.), separated by distances of the order of nanometers. Receiving circular apertures 552 are shown on a conducting cylinder 553, for the propagating microwave radiation stream 404. A base 554 of the cylinder 553 (base shown transparent) forms, with a second base (not shown), a resonant cylindrical cavity.

FIG. 5d shows a preferred embodiment of the microstructure (e.g., anthrax) detector utilizing circular apertures facing into a resonant cavity. The general layout is similar to a preferred embodiment shown in FIG. 4a. In the present preferred embodiment, as shown in FIG. 5d, letter or package mail 401 is carried along on a conveyor belt 402. A propagating microwave radiation stream 404 is shown. Only a few of the circular apertures 551 are shown. These circular apertures 551 are the order of nanometers (1×10⁻⁹ m.), separated by distances of the order of nanometers. Receiving circular apertures 552 are shown on a conducting sphere 554, for the propagating microwave radiation stream 404. An opening 555 in the sphere 554 with a second opening (not shown) interrupt an otherwise continuous resonant spherical cavity to provide an entrance and exit for the mail 401 conveyor belt 402.

Embodiments of the structure of the radar may utilize nanotechnology. Holes or slits (narrow dimension) of the order of 1 nm (1×10⁻⁹ m) may be formed in the walls of waveguides, with spacing of the order on 1 nm, all in relatively regular placement (alignment to within a fraction of 1 nm).

In each of these embodiments, either a monostatic or bistatic arrangement of the transmitting and the receiving apertures may be utilized. Where a bistatic arrangement is used, the beam is progressively scanned over the receiving apertures, repetitively, to achieve a high signal to noise ratio which is proportional to n, the number of “hits” or samples (reflections) from a target. Each scattering of the beam by a target feature may be detected at all other receiving apertures. Time of arrival, phase, amplitude and beam pulse characteristics may be detected. The assembly of this information into a coherent “picture” or visualization is understood in the art. A target recognition software program or hardware/software neural network/matched filter target or target feature recognizer may be applied to the “picture” to ascertain the presence or absence of targeted microstructures, e.g., anthrax spores, brain cells, cancer cells, and/or viruses. The radar part of the microstructure (e.g., anthrax) detector may detect features to 0.1 to 0.01 the gross size of a microstructure (e.g., anthrax spore).

Another preferred embodiment generates a suitable propagating electromagnetic beam utilizing electromagnetic radiation stimulated by impinging light on an appropriate electrically biased semiconductor. Froberg, H., M. Mack, B. B. Hu, X.-C. Zhang and D. H. Auston (Appl. Phys. Lett. 58 (5) 4 Feb. 1991, pp. 446-448) demonstrated that when an array of short photoconducting dipole antennae are illuminated by a train of properly spaced ultrashort optical pulse, the array emits a submillimeter wave beam which can be electrically steered by varying the periodicity of the voltage bias applied to the individual antenna elements. Terahertz (10¹² Hz) radiation (sub-picosecond pulses) have been generated from large aperture Si p-i-n diodes under different biases by femto-second optical impulses. (L. Xu, X.-C. Zhang and D. H. Auston, Appl. Phy. Lett. 59 (26) 23 Dec. 1991, pp.3357-3359) The amplitude and spectral bandwidth of the radiated pulses increased with the reverse bias on the p-i-n diode. The large-aperture p-i-n diode is able to produce a higher bias field (about 140 kV/cm with a lower bias voltage (about 40 V) compared with a large-aperture planar photoconducting antenna which has a bias field of a few kV and requires a high-voltage power supply (op. cit., 3359).

Other prior research include: Time-division multiplexing by a photoconducting antenna array, Froberg, et al., Appl. Phys. Lett. 59 (25) 16 Dec. 1991, pp. 3207-3209; Terahertz pulse propagation in the near field and the far field, Gurtler, et al., J. Opt. Soc. Am. A, 17 (1), pp. 74-83.

In this preferred embodiment, solid state emitters are used in place of the apertures and slits. For example, nanoscale cylindrical-shaped voltage-biased p-i-n diodes may replace round apertures.

FIG. 6a shows the basic mechanism of p-i-n diodes 601 operated by light 602 which results in millimeter/sub-millimeter electromagnetic radiation 603. For example, if p-i-n diodes 601 replace nanoscale apertures wherever they appear on FIGS. 5b, 5c and 5d, then the electromagnetic radiation may be emitted by beam forming phased arrays when stimulated by the appropriate light 602. A plane 604 of, or matrix 604 for, the pin diode 601 array is shown. The actual size of apertures and p-i-n diodes 601 is or the order of nanometers and very dense, while only a few “macro” apertures or p-i-n diodes are actually shown in any of the FIGS. 1-6. Femtosecond optical impulses 602 generate radiated electromagnetic pulses 603 whose amplitude and spectral bandwidth are increased with the reverse bias on the p-i-n diode 601.

FIG. 6b shows the application of light operated p-i-n diodes to utilize millimeter/sub-millimeter electromagnetic radiation for detection of microstructure (e.g., anthrax) and other microstructures. In the present preferred embodiment, as shown in FIG. 6d, letter or package mail 401 is carried along on a conveyor belt 402. A transmitting matrix 654 holds an array of transmitter p-i-n diodes 642. A corresponding receiving matrix 655 holds an array of receiver elements 642. Such receiver elements 642 may include radiation-damaged silicon-on-sapphire photoconductors (Cf. Froberg, et al., 500 GHz electrically steerable photoconducting antenna array, Appl. Phys. Lett. 58 (5), 4 Feb. 1991, pp. 447), free-space electro-optic sampling (cf. Wu and Zhang, Free-space electro-optic sampling of terahertz beams, Appl. Phys. Lett. 67 (24) 11 Dec. 1995) and low-temperature grown GaAs subpicosecond photoconductive switches (cf. Goyette et al., Femtosecond demodulation source for high-resolution submillimeter spectroscopy, Appl. Phys. 67 (25) 18 Dec. 1995, pp. 3810-381). A micromachined low-temperature-grown probe for picosecond photoconductive sampling has is described by Lee et al. (A micromachined photoconductive near-field probe for picosecond pulse propagation measurement on coplanar transmission lines (IEEE Journal on Selected Topics in Quantum Electronics, 7 (4) July/August 2001).

Interferometry techniques may be utilized to improve sensitivity (Interferometric imaging with terahertz pulses, Johnson et al., IEEE Journal. in Selected Topics in Quantum Electronics, 7 (4) July/August 2001, pp. 592-599).

Direct probing with terahertz (THz) electromagnetic radiation may achieve penetration of some barriers. The direct resolution will be limited to 30 to 3000 microns. If we wish to probe target feature scales (L) of 30 to 300 microns in length, one ordinarily wants to probe with electromagnetic radiation a fraction of the feature scale. Thus, using a probing wavelength (λ) of 10% or less (λ<L/10) of the target feature sizes, wavelengths of 3 to 30 microns might be tried for the 30 to 300 microns sized features, respectively. In fact, by decreasing the probing wavelength further (e.g., (λ<L/100) we would discriminate more details. The absorption of these short waves increases rapidly as the wavelength decreases in substances such as wood, concrete, paper, cardboard, water and living tissue.

The absorption goes as: P=P₀e^{-α}, where α=4πκ/c, c is the speed of light in a vacuum, α is the absorption coefficient, and κ is the extinction coefficient, where the complex index of refraction is n=n−iκ.

TABLE I
Penetration Depth in Muscle (measured)
1/α vs. microwave wave length)

| 1/α | λ |
|---|---|
| 0.1 cm | 3 cm |
| 1 cm | 10 cm |
| 10 cm | 3000 cm |

Probing into a concrete wall, through the wall of a room of a house, or into a letter or package, however, is easily done using longer wavelengths, such as millimeter (or shorter) or centimeter wavelength (or longer) microwaves. Such electromagnetic radiation will easily penetrate letters and packages, as well as more substantial barriers.

Other embodiments of this invention include scanning the brain and the body using wavelengths appropriate to the extinction coefficients and safety of the tissue being scanned.

### Anthrax Detector

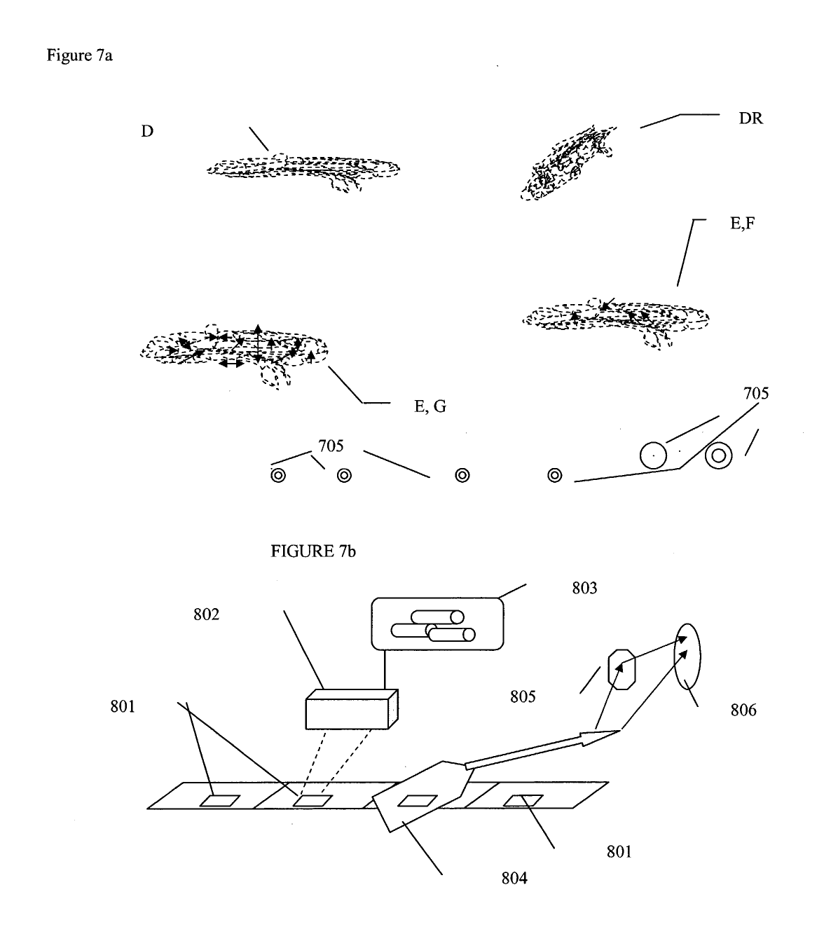

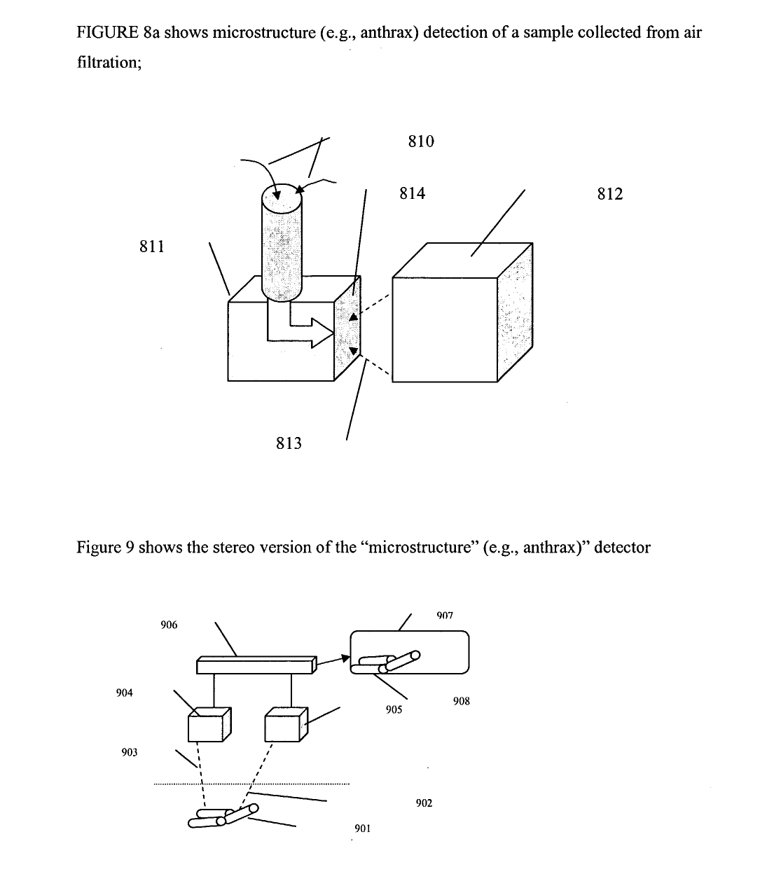

FIG. 7a shows the composite visual presentation 702 of microorganism structure D. The microorganism electrical activity E, if any, and details of any matched-filtered structure may also be shown selectively F (for a selected region or regions of the microorganism) or in entirety G (for the whole microorganism). A rotated view of D is shown in the display element DR. The display may have a capability for rotation of a view on different axes to display information in different aspects and views. Display controls are shown in a schematic manner as 705. The actual controls may be foot-operated. Additionally, control and presentation of views may be carried out at a computer workstation. Various methods for producing useful displays are known in the art. Additional software functionality may be added to the display capability. For example, automatic measurement of lengths, diameters, total numbers and other properties may be displayed automatically from pattern recognition algorithms known in the art. Additionally, absolute coordinates can be calculated and displayed, as well as coordinates relative to a structure or structures in a microorganism A.

FIG. 7b shows a letter/package 801 scanned by scanner 802 for anthrax with the probability of anthrax) detected displayed on the display 803 together with any structure discernable and automatic letter encapsulation 804 and diversion 805 to either observation chamber 805 (to examine anthrax) spores for virulence and for origin) or irradiation chamber 806 (to kill anthrax).

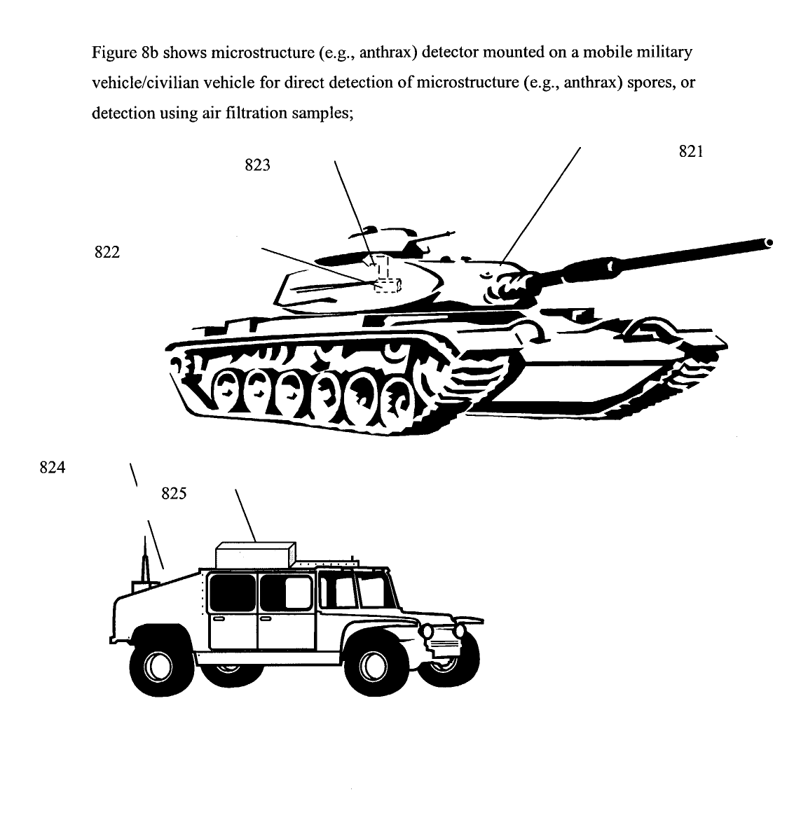

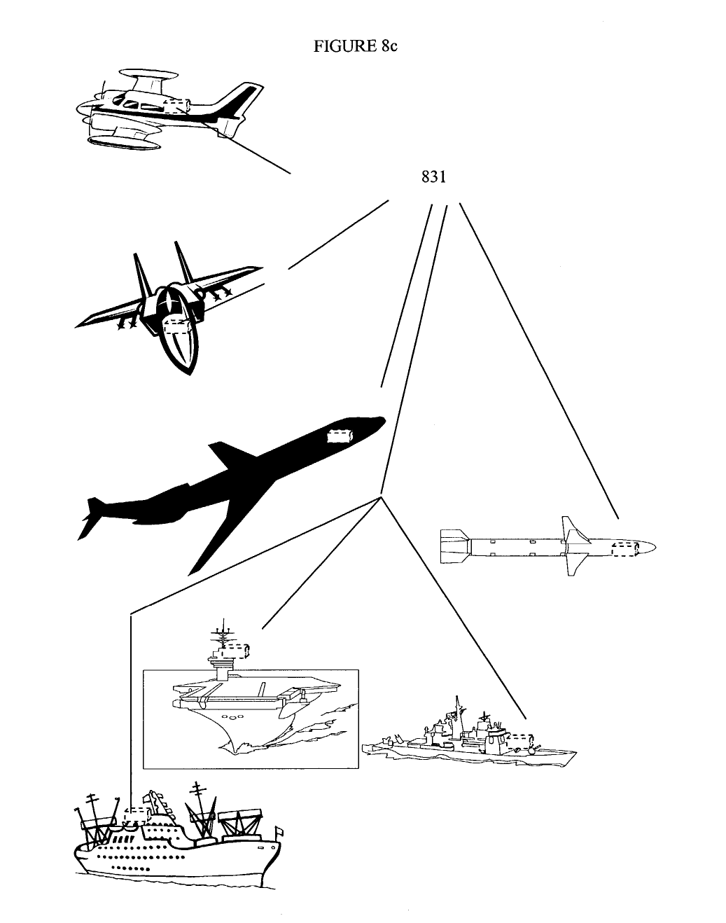

FIG. 8a shows anthrax detection of a sample collected from air filtration. An air filtration collector 811 brings in air 810 which is collected on the surface of a filter 814. The filtrate on the filter surface 814 is then examined by the anthrax detector 812 using at least electromagnetic radiation 813.

FIG. 8b shows anthrax detector 822, 825 mounted on a mobile military vehicle/civilian vehicle for direct detection of anthrax spores 825, or detection using air filtration samples 823, where vehicle may be sealed for ABC warfare.

FIG. 8c shows detector 831 mounted on manned and unmanned aircraft, sea craft, and unmanned missiles for detection of anthrax or other agents with a known signature.

FIG. 9 shows the stereo version of the microstructure (e.g., anthrax) detector. The microstructure (e.g., anthrax) spores or other target material 901 are detected/observed by electromagnetic radiation 902, 903 with a “left” detector 904 and a “right” detector 905 with ancillary electronics 906 for forming a stereoscopic view. A display 907 may indicate detection or no detection, and provide a stereoscopic view, or representation of such, if available.

The issues of what interaction and scattering processes occur for a given microorganism or spore may be quite complicated. Each type of possible interaction may be modeled at different scales, for different combinations and layers of dielectric constants, conductivity, and ionic conduction state, if present.

In order to avoid having to construct such a model, instead one observes to changes which occur as a result of external and internal stimuli to and from the microorganism A. This allows one to analyze the electromagnetic activity, if any, of the microorganism A.

This involves a “null” procedure. Additional measurements and calculations may be achieved for layered papers, such as paper in an envelope, with or without powder, anthrax spores or other agents, both benign and virulent by methods known in the art.

Similarly measurements and calculations with acoustic waves focused on the “target” may be accomplished, by methods known in the art.

### Brain Scanner

In the following description and in all of the cited figures, no part of an animal or human is part of this invention. For example, a human brain may be depicted by dotted lines, to show generally the relative placement of the parts of the invention. The depicted brain is specifically not part of this invention.

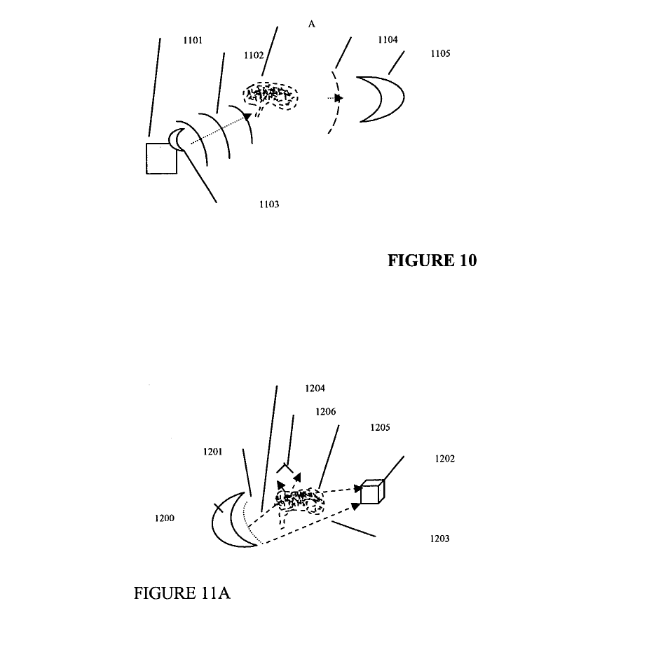

One preferred embodiment of the invention is shown in FIG. 10. A microwave transmitter 1101 transmits a microwave signal 1102 through a suitable antenna 1103. The signal is emitted from the antenna 1103 and is scattered and absorbed by the brain A. A portion of the scattered microwave signal 1104 is collected by a receiving antenna 1105 which is located at some coordinates, xᵣ, yᵣ, zᵣ.

The microwave signal 1102 may be at a fixed frequency, i.e., a fixed wavelength. There may be a range of frequencies in the transmitted signal 1102. In choosing a single frequency, design factors include (a) depth of penetration into tissue of interest as a function of frequency, resolution, which may be proportional to wavelength, system sensitivity as a function of wavelength, since the signal detectability depends upon system sensitivity. Possible multipath problems must also be dealt with. Also a general safety requirement of maintaining radiation levels at least less than 10 mW cm⁻² must be maintained.

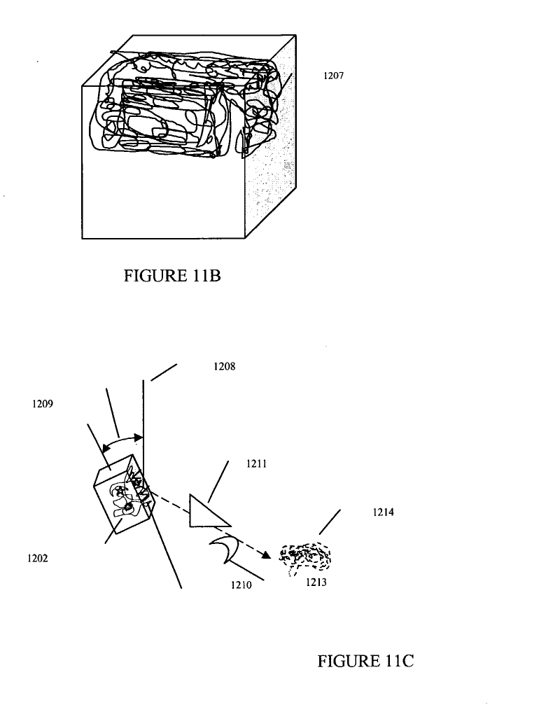

A second preferred embodiment (FIG. 11A) utilizes a microwave signal 1201 from an antenna 1200 to achieve a holographic effect (i.e., acting as a Vander Lugt filter), recorded on an electro-active crystal 1202 such as lithium niobate (LiNbO₃). A portion 1203 of a microwave beam 1201 is recorded directly by the crystal 1202, acting as a reference source. A second portion 1204 of the microwave beam 1201 passes through the brain A and undergoes scattering. A part 1205 of the scattered beam 1206 is recorded by the same electro-active crystal 1202 as the reference beam 1203.

The two microwave beams 1203, 1206 recorded by the electro-active crystal 1202 interfere with each other so as to produce a holographic pattern 1207 (FIG. 11B) in the electro-active crystal 1202.

The electro-active crystal 1202 is read out with a coherent light source 1208 by shining the light at a “glint” angle 1209 to the surface 1210 of the crystal 1202. The glint angle 1209 should not exceed the Brewster angle or total reflection will occur.

However, at a suitable glint angle 1209, the coherent light source 1208 encounters the microwave induced holographic pattern 1207 as a much tightened structure, able to appropriately diffract the coherent light source 1208. The “foreshortening” of the holographic pattern 1207 may be corrected by an aberration correction unit 1211, reflection by an appropriate convex mirror 1213 or other transform methods. The coherent light source illumination of the holographic recording crystal will give rise to a holographic presentation 1214 of the scanned brain A (FIG. 11c).

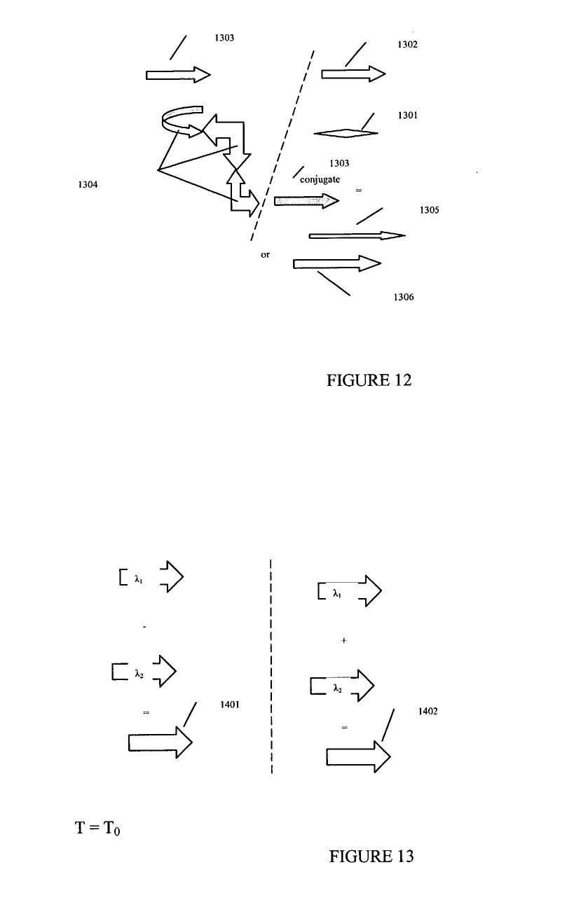

Time differencing 1301 (FIG. 12) or spatial differencing 1401 (FIG. 13) may be used to recover information from a large number of neurons, of the order of 1×10¹⁰. Observations of changes in the state of a neuron from conducting to non-conducting or vice versa are of extreme importance. For example, the change in neuron states from an environment of silence to a particular audio frequency would be of diagnostic and research interest. Similarly, the change in neuron states from a subject viewing different patterns or colors, or being exposed to different odors.

The differencing 1301 (FIG. 12) may be done by overlaying a picture of the neuron states at one moment 1302 with that of a time delayed picture 1303. This may be accomplished by sending the first picture 1303 through a time delay light pipe 1304 and retrieving at a time to overlay the second picture 1302, with the first picture 1303 conjugated to be the visual equivalent of the negative its original self, −S(t₁), so that the difference picture is S(t₂)−S(t₁) 1305,. That is, a differenced state, or alternatively, a direct interference state is produced, S(t₂)+S(t₁) 1306. The difference or interference state may be a hologram.

Another form of the differencing or interference state may be produced by differencing or interfering states derived from different wavelengths passing through the brain A at the same time. The difference is S(λ₁)−S(λ₂) 1401 (FIG. 13), while the interference state is S(λ₁)+S(λ₂) 1402.

For some cases, λ₂=λ₁+δλ, where λ₁, λ₂ are different wavelengths and where δλ is a small difference of wavelength.

A lower resolution may allow averaging over a volume of neurons, showing less detail, but providing, in some cases, an easier to understand picture of neural activity.

The detector of microwave source may have a resolution of

$$δ = \frac{λ}{2 {L}_{eff}} R = \frac{λ \cdot RNd cos {θ}_{0}}{2 \cdot 0.8858 R λ} = \frac{Nd cos {θ}_{0}}{1.7716}$$

If a 10 mn to 100 nm resolution is utilized, a resolution of 0.01 to 0.1 μm may be achieved. A typical mammalian nerve axon diameter is 1 μm-20 μm.

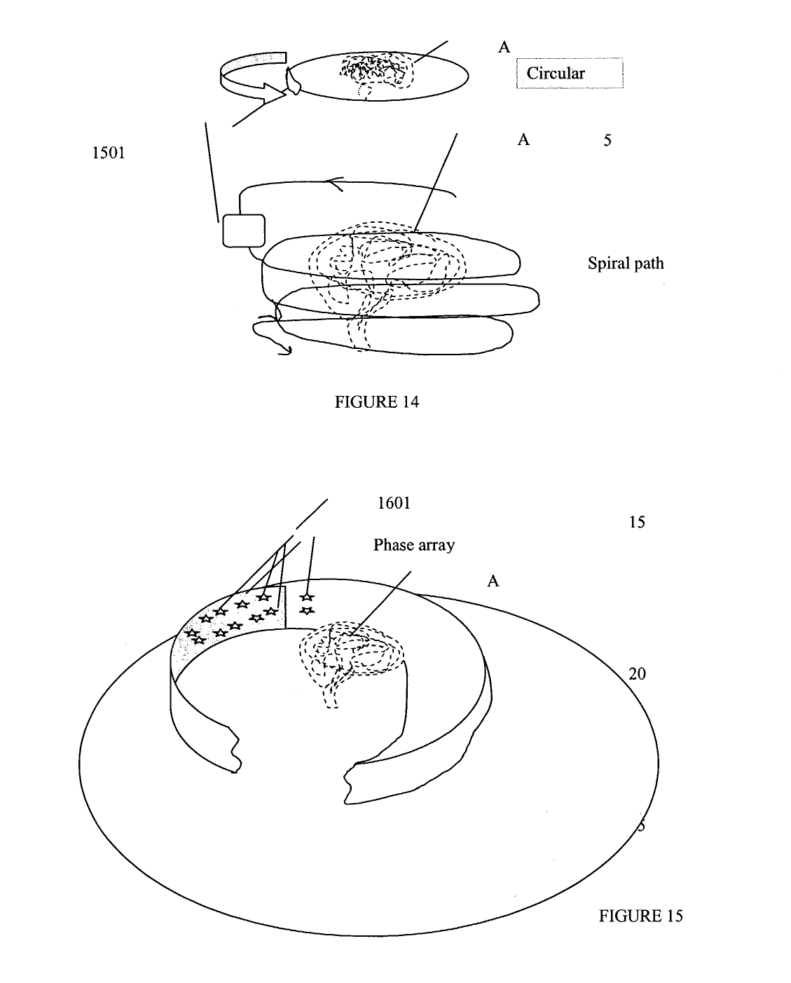

A moving antenna 1501 (in a circular, spiral or other pathway around the brain A) (FIG. 14) may be used for the synthetic aperture; or, a stationary phased array 1601 (FIG. 15) maybe used.

In order to best penetrate the brain tissue with a relatively low energy and avoidance of health risk, a wavelength of up to 10 cm to 1 m may be used. The resolution of a scan is no longer dependent on matching a very short wavelength to the order of the object being resolved, which is approximately 5 μm, in this example. This alleviates many problems of system sensitivity and too high microwave power levels.

Processing for the synthetic radar type of source may be accomplished electronically and/or optically, as is known in the art. The processing procedure may incorporate the differencing and interference states as above.

The issues of what interaction and scattering processes occur at each neuron are quite complicated. Each type of possible interaction may be modeled at different scales, for different combinations and layers of dielectric constants, conductivity, and neural conduction state, with various actions occurring in a neuron's sodium, potassium and chloride channels, as well as for the total axial (longitudinal) currents inside the axon and outside the axon.

In order to avoid having to construct such a model, instead one observes to changes which occur as a result of external and internal stimuli to and from the brain A. This allows one to analyze the electromagnetic activity of the brain A.

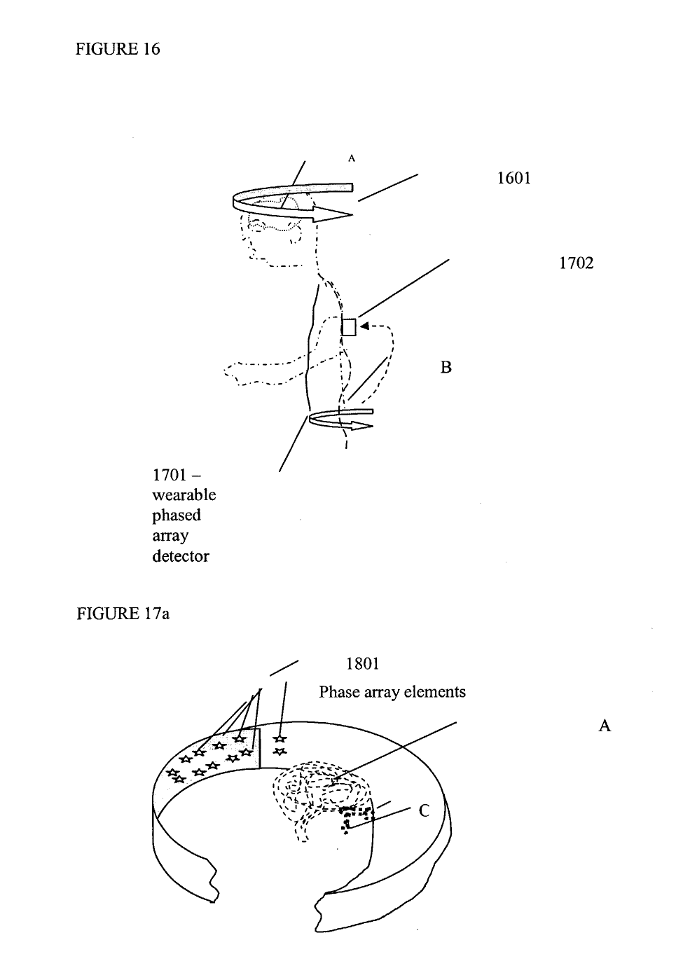

FIG. 16 shows an application of the synthetic aperture remote sensing 1701 applied to detecting information in a nerve or nerve bundle of the vagus nerve B which tells the glucose level to the brain A. It is shown with a feedback loop to an insulin pump 1702. The apparatus 1701 detects the information and through an intermediate computer or electronic chip, activates the insulin pump as needed.

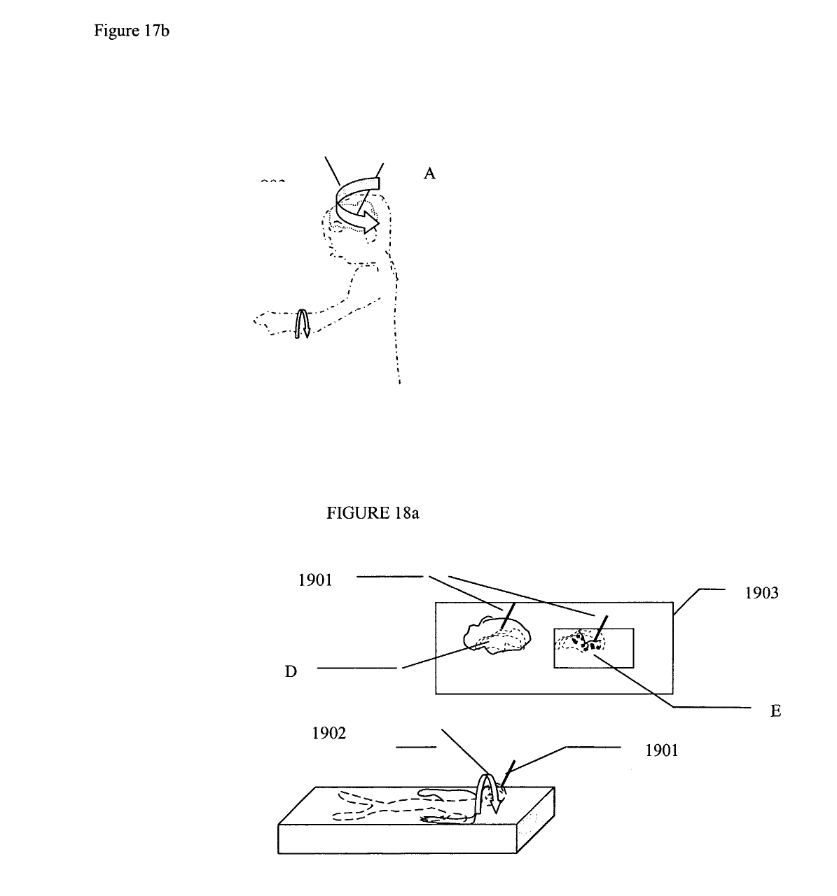

FIG. 17a shows an application to the correlation of vision, i.e., optical nerve information, with brain activity. This can extend to other types of correlation including aural-brain, aural-lingual, manual-brain, manual-lingual and so on. FIG. 17A shows one detector array 1801 for both brain and optic nerves C. FIG. 17b shows two phased array detectors 1802, 1803, one for arm and hand nerves 1802 and the other for the brain 1803. Not shown is the analysis computer and signal processing elements and chips and ancillary equipment, which may include digital recorders for later analysis.

### Stereotactic Probe Imaging

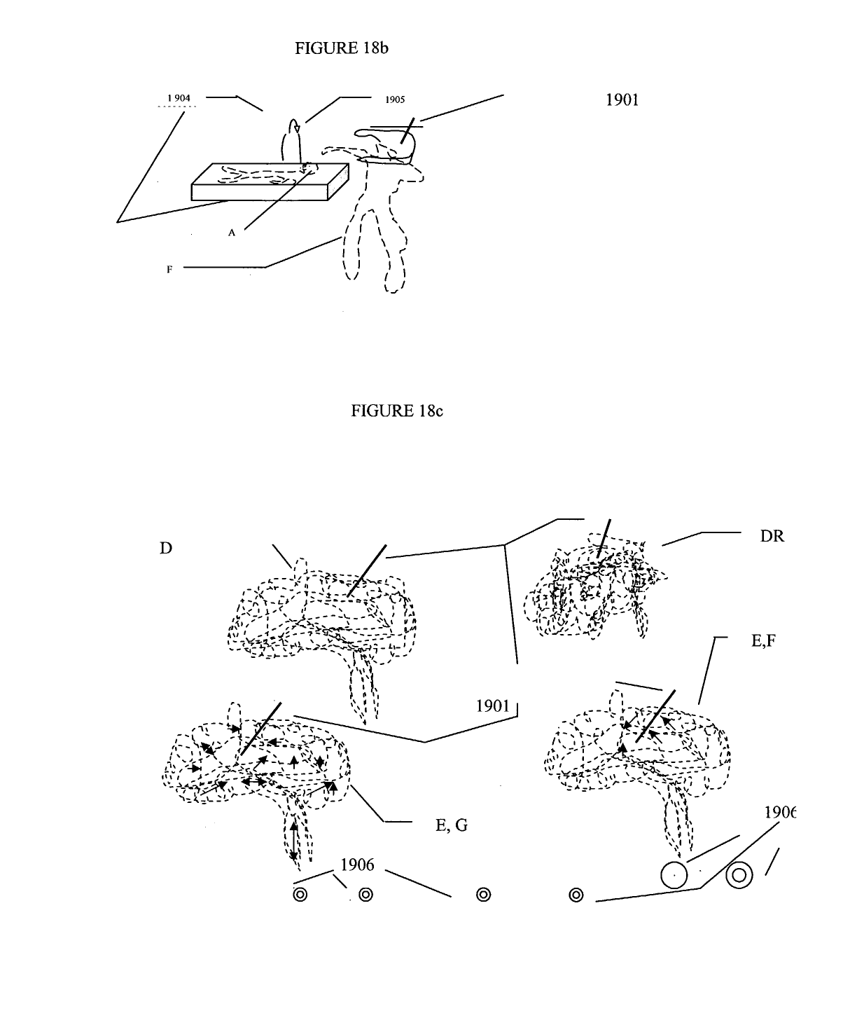

The viewing and detecting aspect of this invention are show in FIGS. 10-17B and the discussion related to these Figures. The detection aspects principally focus on detected brain structure relative to an imbedded probe 1901 (FIGS. 18A, 18B, 18C, below). However, knowledge of the electrical activity in neurons or neuron bundles may remain important while conducting a neurosurgical procedure.

FIG. 18A shows a stereotactic probe 1901 in an embedded position in a brain A, such as that of a patient undergoing a neurosurgical procedure. A stationary transmitter-detector phased array radar 1902 detects the detailed brain structure D, the brain electrical activity E and the stereotactic probe 1901 and provides a composite visual presentation 1903 in real time.

FIG. 18B shows a synthetic aperture radar 1904 with a moving antenna 1905 as the electromagnetic transmitter-detector. The moving antenna 1905 is placed out of the way of a neurosurgeon F.

FIG. 18C shows the composite visual presentation 1903 of brain structure D and stereotactic probe 1901. The brain electrical activity E may also be shown selectively F (for a selected region or regions of the brain) or in entirety G (for the whole brain). A rotated view of D is shown in the display element DR. The display may have a capability for rotation of a view on different axes to display information in different aspects and views. Display controls are shown in a schematic manner as 1906. The actual controls may be foot-operated. Additionally, control and presentation of views may be carried out by an assistant to the neurosurgeon at a computer workstation at the voice commands of the surgeon. Various methods for producing useful displays are known in the art. Additional software functionality may be added to the display capability.

The stereotactic probe 1901 may be visually located on the display 1903. Additionally, absolute coordinates can be calculated and displayed, as well as coordinates relative to a structure or structures in a brain A.

The stereotactic probe may be made of a metal, of metals, or of a composite metal and plastic (or, polymer), or of a plastic or polymer. The stereotactic probe may contain at least one instrument known in the art including miniaturized Doppler ultrasound, miniaturized camera for visible and/or infrared light, miniaturized light and miniaturized piezoelectric pressure sensor. A requirement of the probe is that it be visible to the electromagnetic detector, if not in whole, at least in part.

### Cancer Cell/Virus Virulon Detection and Elimination

An embodiment of this invention can scan a human body for and detect and destroy cancer cells which are susceptible to elevated temperatures.

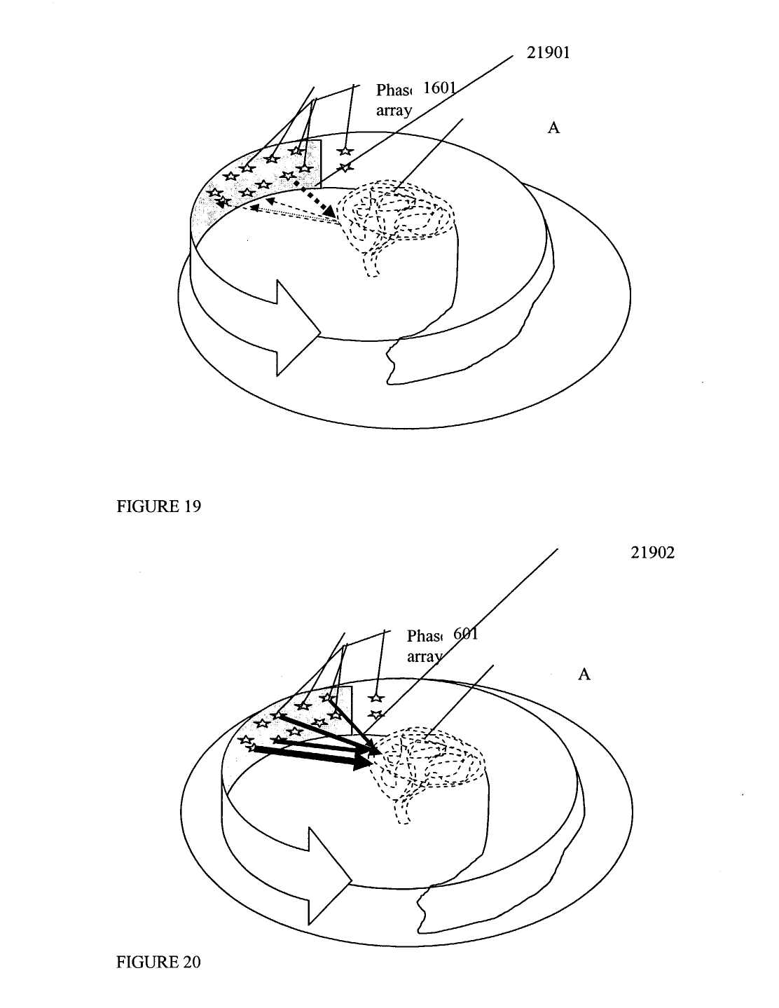

This embodiment is similar to the microstructure (e.g., anthrax) detector or the brain scanner in that it is set up to detect cancer cells on the same order of magnitude as the microstructure (e.g., anthrax) detector and brain scanner. In FIG. 19, the interrogating electromagnetic radiation 21901 having spotted a cancer cell, based on its target detection module, the detector acts to focus many electromagnetic beams 21902 from all angles (FIG. 20).

The power in each eliminator 21902 beam may be boosted above the ordinary interrogation power. It acts to adaptively form beams to irradiate the cancer cell and raise its temperature. For example, cells will die when their temperature is raised to 61 ° C. for a time of 1 second. Since the local area in ordinary living tissue will take milliseconds to undergo cooling, a large number of beams converging on a given volume of cell or cells being treated may be cycled so as to be able to examine, detect and treat a larger volume of living tissue.

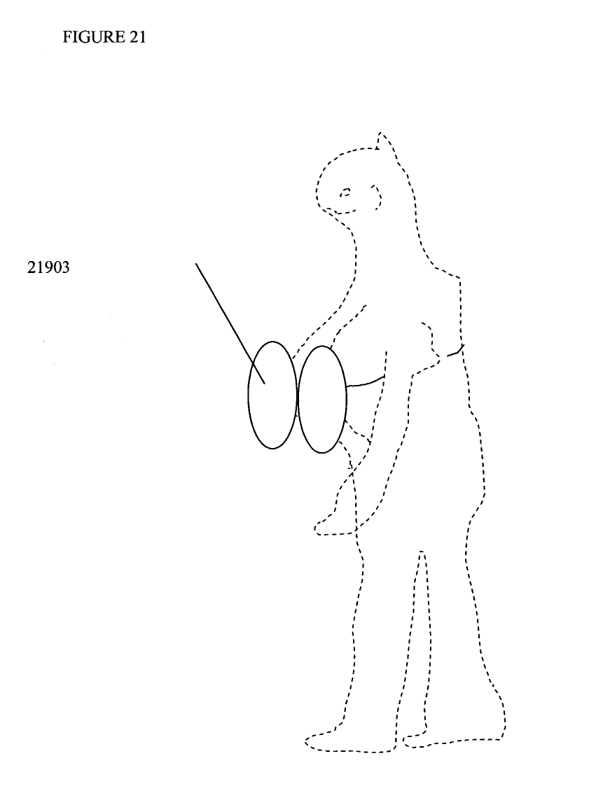

For certain areas of the body, a cancer-detector-eliminator may be worn, such as a bra for detecting and preventing breast cancer 21903 (FIG. 21). The bra 21903 will have to adaptively keep track of where its eliminator beams 21902 are, in real time, as the wearers body may be in motion.

### Detection of Weapons and Explosives

An embodiment of this invention comprises a method and apparatus for detection of weapons and explosives.

In order to best penetrate mailed packages or letters or objects or human tissue with a relatively low energy and avoidance of health risk, a wavelength of up to 1 m to 2 m may be used. The resolution of a scan is no longer dependent on matching a very short wavelength to the order of the object being resolved, which is approximately 1 μm. This alleviates many problems of system sensitivity and too high microwave power levels. The wavelength may be chosen shorter, however, according to the best requirements of a given application. Instead of a “half-wavelength” or a “quarter wavelength” antenna, a ½ⁿ wavelength antenna may be required, where n is a relatively large integer. Also, for example, a millimeter waveguide may be provided with a slot or hole or microfabricated emission source.

Processing for the “phased array-synthetic aperture radar” type of source may be accomplished electronically and/or optically, as is known in the art. The processing procedure may incorporate the differencing and interference states as above.

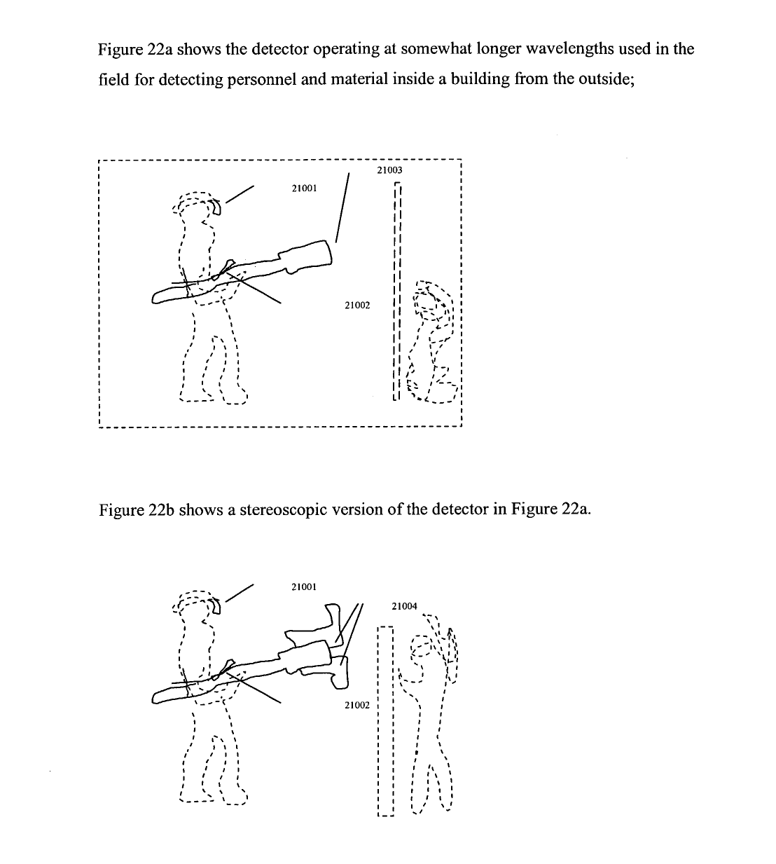

FIG. 22a shows the detector operating at somewhat longer wavelengths used in the field for detecting personnel and material inside a building from the outside. The wavelengths used here are longer, since the thickness of the wall to be penetrated by the electromagnetic radiation is thicker than the walls of envelopes and mailed packages. However, the method of operation is the same as the microstructure (e.g., anthrax) detector, except scaled up.

A dotted soldier is shown with the “privacy invader”, i.e., the scaled up microstructure (e.g., anthrax) detector 11003. Also shown is a head down display 11002 and a heads-up display 11001.

FIG. 22b shows a stereoscopic version of the detector in FIG. 19A. The heads-up display 11001 and the heads down display 11002 are shown with a “stereo-detector” 11004. The device may be operated by an umbilical cord electrical connection or it may be battery operated, where the batteries may be rechargeable on a vehicle in the field).

### Other Embodiments

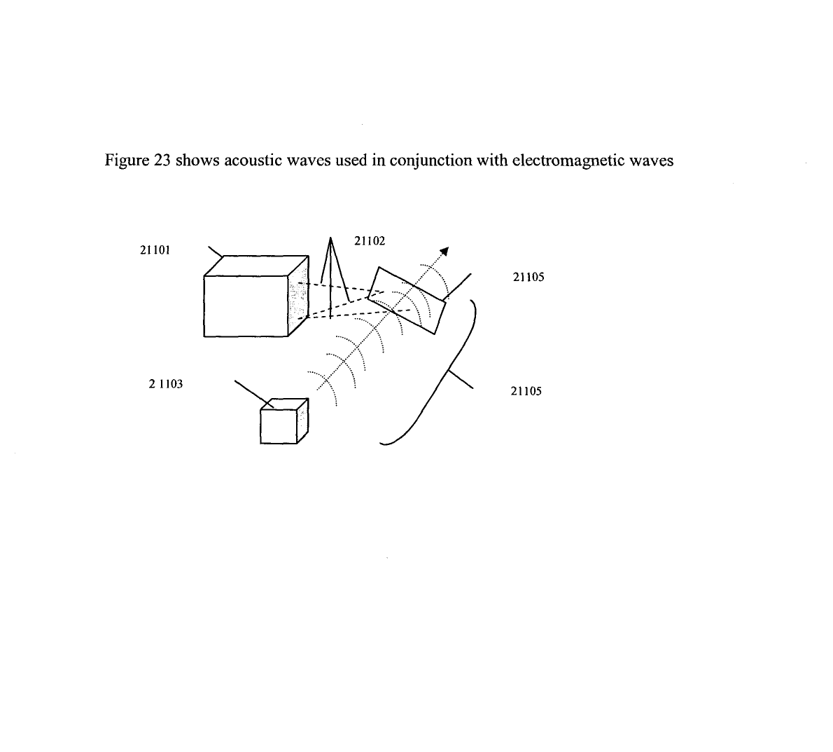

FIG. 23 shows acoustic waves used in conjunction with electromagnetic waves. The electromagnetic detector 21101 interrogates a target with electromagnetic radiation 21102, which may be an envelope 21105 while an acoustic wave generator 21103 shines an acoustic beam 21105 on the target. Several enhanced detection features may result. The first is that motion of a powder may occur and so be detected. The second is that the scattering may be sufficiently different from different materials, so as to have an additional signature due to the change in dielectric permeability resulting from deformation of a solid body.

Other embodiments of this invention include a large scale structure oriented for detecting features on planets outside our solar system.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention.

What is claimed is:

1. An penetrating electromagnetic detector, for examining a target in detail, comprising:
(a) electromagnetic radiation of a wavelength chosen for ease of penetration into volume to be viewed;
(b) a beam forming device for the electromagnetic radiation;
(c) detectors of electromagnetic radiation, scattered by target;
(d) analysis modules to reconstruct image of target from scattered electromagnetic radiation, whereby said target is scanned and viewed in detail.

2. The detector of claim 1, further comprising a transmitter phased array, with ability to scan over target.

3. The detector of claim 1, further comprising system means to examine anthrax in mail envelopes and packages.

4. The detector of claim 1, further comprising system means to examine brain cells in vivo.

5. The detector of claim 1, further comprising system means to examine stereotactic instruments during neurosurgery as well as the brain cells in proximity to the stereotactic instruments.

6. The detector of claim 1 further comprising system means to detect and eliminate cancer cells and cells containing viruses.

7. The detector of claim 1 further comprising system means to detect weapons and explosives.

8. The detector of claim 1 further comprising system means to see through walls.

9. 10. The detector of claim 1 further comprising system means to detect utilize ultrasonic means to provide enhanced detection and analysis of some targets.

10. The detector of claim 1 further comprising system means to detect distant planets not in our Sun's Solar System.

11. An intermediate detection method, comprising the steps of:
a. utilizing a microwave signal from a microwave source;
b. sending part of microwave signal through brain;
c. recording signal which has passed through brain along with reference signal which comes directly from microwave source;
d. recording said signals on a photo-active crystal.

12. The intermediate detection method of claim 11, further comprising the steps of:
a. shining a coherent light source on the photo-active crystal at some glint angle less than the Brewster angle;
b. reading out the foreshortened holographic pattern by visible light.

13. A method for comparing the brain states at two times close in time, comprising:
a. delaying signal which represents the brain state occurring first in time
b. comparing the two brain states by subtracting one state from the delayed state of the other.
